(19) United States
(12) Patent Application Publication
(10) Pub. No.: US 2025/0299588 A1
(43) Pub. Date: Sep. 25, 2025

(54) SYSTEM FOR PERSONALIZED HEALTH MONITORING AND IMPROVEMENT

(71) Applicant: Zoe Limited, London (GB)

(72) Inventors: Sam Aydin Berkay, London (GB); Christopher Clive Bonnett, London (GB); Julien Pascal Lavigne du Cadet, London (GB); Daniela Genkova Zheleva, London (GB); Rhea Eve DSouza, London (GB); Anamaria Gartmann, London (GB); Joseph Arthur Simmons, Lisbon (PT); Jon Alberdi, Hendaye (FR); Konstantinos Frantzis, London (GB); Carmen Rodriguez Gonzalvez, Sesto Calende (IT); Luke William Matthews, Birmingham (GB); Matthew James Hanley, Bristol (GB); Arnab Pushilal, London (GB); Ramiro Alejandro Toro Guerrero, Barcelona (ES); Viktoriya Chavdarova Georgieva, Macclesfield (GB); Laura Anne Mahy, London (GB); Diane Maëliss Gasselin, Aix-en-Provence (FR); José María Ruiz Aguilera, London (GB); Francisco Maria Trigo da Roza Rocha da Silva Carvalho Araújo, Lisbon (PT)

(21) Appl. No.: 18/649,265

(22) Filed: Apr. 29, 2024

Related U.S. Application Data
(63) Continuation of application No. 18/699,741, filed on Jan. 1, 0001.

(51) Int. Cl. G09B 5/02; A61B 5/00; G16H 10/20; G16H 20/60

(52) U.S. Cl. CPC G09B 5/02; A61B 5/743; A61B 5/7475; G16H 10/20; G16H 20/60

(57) ABSTRACT

A system for generating personalized health food scores for one or more participants and/or group of participants (such as a household) based on participant data including microbiome data of each participant. In some cases, the system may generate recommended meal plan options based on the available food items, the participant data for each member of a group.

### CROSS REFERENCE TO RELATED APPLICATIONS

This application is a continuation of and claims priority to U.S. application Ser. No. 18/699,741, filed on Apr. 9, 2024 and entitled “SYSTEM FOR PERSONALIZED HEALTH MONITORING AND IMPROVEMENT,” which is a U.S. national stage application under 35 USC § 371 of International Application No. PCT/EP2024/057324 filed on Mar. 19, 2024,” the entirety contents of which is incorporated herein by reference.

### BACKGROUND

Today, many companies offer services for improving health of an individual or family via pre-planned meals or pre-planned recipes. Typically, these services provide at home meal delivery or weekly notifications of recipes and associated shopping lists. Unfortunately, these services are often fixed for all participants and fail to account for shopping habits, locally available produce and products, and provide no customization based on the personal biology of the participant.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is an example block diagram of a system for providing personalized and group health monitoring and meal planning, according to some implementations.

FIG. 2 is a flow diagram illustrating an example process associated with the system for providing personalized and group health monitoring and meal planning of FIG. 1, according to some implementations.

FIG. 3 is a flow diagram illustrating an example process associated with the system for providing personalized and group health monitoring and meal planning of FIG. 1, according to some implementations.

FIG. 4 is a flow diagram illustrating an example process associated with the system for providing personalized and group health monitoring and meal planning of FIG. 1, according to some implementations.

FIG. 5 is a flow diagram illustrating an example process associated with the system for providing personalized and group health monitoring and meal planning of FIG. 1, according to some implementations.

FIG. 6 is an example system for providing personalized and group health monitoring and meal planning that may implement the techniques described herein according to some implementations.

FIG. 7 is an example user device that may implement the techniques described herein according to some implementations.

FIG. 8 is an example pictorial diagram illustrating an example user interface associated with a personalized health food score of a participant according to some implementations.

FIG. 9 is an example pictorial diagram illustrating an example user interface associated with personalized recommendations to improve the participant's health food score according to some implementations.

FIG. 10 is an example pictorial diagram illustrating an example user interface associated with setting a personalized target for a given period of time according to some implementations.

FIG. 11 is another example pictorial diagram illustrating an example user interface associated with setting a personalized target for a given period of time according to some implementations.

FIG. 12 is an example pictorial diagram illustrating an example user interface of a notification associated with a personalized target for a given period of time according to some implementations.

FIG. 13 is an example pictorial diagram illustrating an example user interface associated with health food score for a meal plan option including each ingredient food score personalized for the participant according to some implementations.

FIG. 14 is an example pictorial diagram illustrating an example user interface associated with generating meal plan options according to some implementations.

FIG. 15 is another example pictorial diagram illustrating an example user interface associated with generating meal plan options according to some implementations.

FIG. 16 is another example pictorial diagram illustrating an example user interface associated with generating meal plan options according to some implementations.

FIG. 17 is an example pictorial diagram illustrating an example user interface associated with boosting meal plan options according to some implementations.

FIG. 18 is another example pictorial diagram illustrating an example user interface associated with boosting meal plan options according to some implementations.

FIG. 19 is another example pictorial diagram illustrating an example user interface associated with boosting meal plan options according to some implementations.

FIG. 20 is an example pictorial diagram illustrating an example user interface associated with managing a group according to some implementations.

FIG. 21 is an example pictorial diagram illustrating an example user interface associated with joining a group according to some implementations.

FIG. 22 is another example pictorial diagram illustrating an example user interface associated with managing a group according to some implementations.

FIG. 23 is an example pictorial diagram illustrating an example user interface associated with generating meal plan options for a given period of time according to some implementations.

FIG. 24 is another example pictorial diagram illustrating an example user interface associated with generating meal plan options for a given period of time according to some implementations.

FIG. 25 is another example pictorial diagram illustrating an example user interface associated with generating meal plan options for a given period of time according to some implementations.

FIG. 26 is another example pictorial diagram illustrating an example user interface associated with generating meal plan options for a given period of time according to some implementations.

FIG. 27 is another example pictorial diagram illustrating an example user interface associated with generating meal plan options for a given period of time according to some implementations.

FIG. 28 is another example pictorial diagram illustrating an example user interface associated with generating meal plan options for a given period of time according to some implementations.

FIG. 29 is an example pictorial diagram illustrating an example user interface associated with generating a shopping list for a given period of time according to some implementations.

FIG. 30 is an example pictorial diagram illustrating an example user interface associated with logging consumption data for a given period of time according to some implementations.

FIG. 31 is an example pictorial diagram illustrating an example user interface associated with a personalized health food score for a given period of time according to some implementations.

FIG. 32 is an example series of pictorial diagrams illustrating an example user interface associated with scanning a receipt and logging consumption data for a given period of time according to some implementations.

FIG. 33 is an example series of pictorial diagrams illustrating an example user interface associated with scanning a receipt and logging consumption data for a given period of time according to some implementations.

FIG. 34 is an example pictorial diagram illustrating an example user interface associated with generating a contextual based meal plan option for a given meal according to some implementations.

FIG. 35 is another example pictorial diagram illustrating an example user interface associated with generating a contextual based meal plan option for a given meal according to some implementations.

### DETAILED DESCRIPTION

Discussed herein are systems, applications, and user interfaces for providing personalized and participant group (such as a household) health monitoring and meal planning. In various implementing, the system discussed herein may include a cloud-based service and an application hosted on a user device for receiving user input and provide recommendations, such as providing meal plans, health-based and nutrition-based advice and recommendations that are personalized based at least in part on the biology (e.g., the microbiome of a participants digestive system, blood sugar and blood fat postprandial responses, age, allergies, physical location, overall health and wellness, other demographic data, and the like) of each participant. In some cases, the recommendations may be personalized for a household unit or other group of participants that shares meals, such as recommendations optimized based on the personal biology of two or more participants that cohabitate with each other.

Accordingly, unlike conventional meal services which provide pre-planned meals or recipes that are designed based on overall health goals and professional advice for the masses, the system discussed herein may make personalized recommendations for the individual or group of participants based on the individual needs and personalized biology resulting in improved health benefits and overall fewer resource consumptions (such as computing resources as results are achieved with fewer iterations).

In some implementations, the system discussed herein, may allow for gradual health improvement based on an original health and eating habits of the individual or group of participants. For example, the system may include an application hosted on a user device that may enable a participant to capture food data associated with grocery shopping receipts, contents of a food storage area (e.g., a pantry, refrigerator, cold storage, freezer, or the like) and to provide the associated captured data to the cloud-based system.

The cloud-based system may include one or more machine learned models that are trained to segment, classify, detect foods and ingredients, and generate data associated with each detected food or ingredient. For example, the one or more machine learned models may be configured to disambiguate between different items, foods, or ingredients within image data of a receipt or food storage area. The one or more machine learned models may classify each of the detected items, such as classifying items between types of foods, such as meats and vegetables, between types of each class (e.g., chicken from pork or the like) as well as between base ingredients of types such as whole grain bread, white bread, wheat bread, or the like. In some cases, the one or more machine learned models may also determine nutritional data associated with each classified item. For example, the system may determine a calorie count, fiber content, fat quantity and quality (e.g., monounsaturated or polyunsaturated, saturated, and the like), carbohydrate quantity and quality (e.g. glycemic index), vitamin content, mineral content, protein type (e.g., plant-based or animal-based) and content, salt/sodium content, water content, level of processing (e.g. NOVA) and the like.

In some examples, the one or more machine learned models for segmenting, classifying, detecting foods and ingredients, and generating data associated with each detected food or ingredient may be trained on image data with associated food data.

The system may also receive participant data associated with a participant. The participant data may include microbiome data, such as microbiome data identifying the presence and/or quantity of various bacteria and the like. For example, the participant data may include lab generated data, such as when a participant provides biological samples to a lab for testing. For instance, a participant may provide a lab with a sample, such as a stool sample, for microbiome analysis. As an example, metagenomic testing can be performed using the sample to allow the DNA of a microbiome of an individual to be digitalized. Generally, a microbiome analysis includes determining the composition and function of a community of microorganisms in a particular location, such as within the gut of a user. An individual's microbiome appears to have a strong causal relationship to metabolism, weight and health, yet only ten to thirty percent of the microbiome is common across different individuals.

The participant data may also include health data, blood data, glucose data, ketone data, nutrition data, genetic data, saliva data, biometric data, questionnaire data, psychological data (e.g., hunger, sleep quality, mood, and the like) as well as other types of data. Generally, health data may refer to any psychological, subjective and/or objective data that relates to and is associated with one or more individuals. The health data might be obtained through testing, self-reporting (such as via the application hosted on the user device), and the like.

In some examples, the health data includes wearable data obtained from technology worn and/or utilized by a participant. For instance, a participant may wear a fitness device, such as an activity-monitoring device, that monitors motion, heart rate, determines how much a participant has slept, the number of calories burned, activities performed, blood pressure, body temperature, and the like. The participant may also wear a continuous glucose meter that monitors blood glucose levels often by measuring levels of glucose in interstitial fluid.

A participant may also provide data that may be utilized to predict the target values and/or changes to the target values and generate the nutritional recommendations using other devices such as blood glucose monitors, finger pricks which in some examples are used with dried blood spot cards, blood pressure monitors, and the like. A participant may also input data into one or more software applications (or provide the data some other way) that may be utilized. For example, a participant may enter the foods the participant consumed during a meal, how much the participant slept, what exercise the participant performed during a given period of time, how hungry the participant is at one or more times of day including mealtimes, how the participant feel, what medication the participant consume, and the like. As another example, a participant or a lab may provide test data determined from one or more tests, such as urinalysis test strips, blood test strips, and the like. The test data may come from different sources, such as but not limited to from one or more of an individual, a lab, a doctor, an organization, and/or some other data source. A participant may also provide data about their food preferences, medical guidance the participant has received, or personalized food constraints/preferences, such as allergies, being vegan, gluten free, keto or other adhered diet, kosher, halal, or the like.

In some implementations, utilizing the health data personalized for a participant and the food data, such as the food data captured from the grocery receipt or food storage area, and the participant data, the system may generate (e.g., via one or more machine learned models, algorithmic techniques, heuristics, or the like) personalized food scores for each food to individual pair. For example, baby kale for a first individual may have a first score while for a second individual the same baby kale may have a second score different than the first score. In this manner, each individual may have a personalized food score for each item (e.g., food, ingredient, supplement, product, and the like). The system may also generate (e.g., via one or more machine learned models, algorithmic techniques, heuristics, or the like) utilizing the health data a personalized list of gut boosters (e.g., positive items for the participants microbiome) and gut suppressors (negative items for the participants microbiome). In some cases, the system may also generate (e.g., via one or more machine learned models, algorithmic techniques, heuristics, or the like) in addition to or in lieu of participant specified preferences, dietary preferences and/or exclusions.

In some case, the system may also generate a time period specific (such as daily, weekly, monthly, and the like) health food scores of the participant based at least in part on the participant data, the food data, and the output of the one or more machine learned models. For example, based on the food data representing a weekly shopping receipt and the participant data personalized for the user, the system may generate an individualized and personalized health food score for the participant representing the value of the purchased items for the individual participant when consumed. In some cases, the health food scores may be based at least in part on the personalized food scores for each food. In some cases, the health food scores may also be influenced, weighted, and/or biased based on time of day consumed (e.g., as input by the participant via the application hosted on the user device), season of the year, geographic location, weather, current health conditions (e.g., presence of a particular disease or sickness), activity level during the corresponding period, presence or absence of gut boosters or suppressors, and the like.

In some cases, in addition to the health food scores for the given time periods, the system may also generate one or more meal or recipe for the user based at least in part on the detected ingredients and each food score for the detected ingredients personalized for each the participant. The system may also generate a meal or recipe score personalized for each individual participant based on the food score for the detected ingredients personalized for each the participant. For example, the system may attempt to balance the meal planning based on the purchased items represented in the image data of a receipt for an overall healthiest possible time period (such as a week). In some instances, the system may present multiple alternative recipes or meal plan options based on the item list to allow the participant a choice of meals during the time period. In some instances, such as when a group of participants or household has two or more participating members, the system may generate the meal plan and recipes (e.g., meal plan options) to provide the highest health food scores across the multiple participants (such as a highest average score, highest median score, highest summed score, highest minimum score, or the like).

In some cases, the system may apply one or more thresholds or metrics for each participant to ensure each participant in a group maintains a time period based health food score equal to or exceeding the thresholds on one or more periods of time (such as for each meal, daily, weekly, monthly and the like). In this manner, the system may ensure that each member of the group or household is benefiting from the meal planning and recipes generated by the system. In some cases, when the system presents, via the application on the display of the user device, choices of recipes for the participants and the participants other group members, the recipe choice may include the recipe score personalized for each member of the group to assist the group in selecting the optimal set of recipes for both health and taste preferences of each participant. In this manner, the system may allow the final selection and health scores to be selected by the subjective preferences of the participants (such as if a group desires to favor the health of one individual over another).

In some cases, the system may generate a food storage area score or overall pantry score for each participant. For example, utilizing the logged time data as well as the food data generated from a weekly receipt or food storage area scan, as well as consumed food data, the system may generate a pantry score associating the health score of the overall pantry of the participant based on the participants personalized health and microbiome. In some cases, the application hosted on the user device may provide data related to changes in the pantry score over time, such as to provide each participant with feedback on how the pantry is improving or diminishing from time period to time period. In this manner, the system may encourage steady improvement of the pantry items, thereby resulting in overall healthier eating for the participant over time. In addition to the pantry score, the system may also generate an ingredients list for each item available for use in the food storage areas of a group. The ingredients list may include a personalized food health score for each participant in the group.

In some examples, the meal planning and recipes may be the output of one or more machine learned models that receive the time period food data and the health data for each participant in the group. For example, the one or more recipes and meal planning machine learned models may be trained on food data and heath data for individual participants and/or groups with various numbers of participants.

In some cases, following the completion of a time period (such as week), the system may utilize the meal plan, recipes, and/or food data represented by a receipt to log the nutritional consumption of each participant during the period of time. In this manner, the system discussed herein reduces error caused by human data entry, reduces time investment of participants (often resulting in greater health benefits and participation by the users), greater flexibility in meal planning, as well as more personalized health data and health results, and the like when compared with conventional meal planning services. In some cases, the system may request user input to confirm consumption of each meal or recipe, or food item. In these cases, the system may filter the food lists to list the meal, recipe or individual food items at the top of for ease of use by the participant. In this manner, the participant does not have to manually search lists of items to identify and input consumed products.

In some examples, the system may also be configured to provide shopping recommendations to the participant. For example, the system may receive the food data and/or receipt data (e.g., sensor data of the receipt) and determine, based on the receipt data and either or both of the participant data and/or the food scores for various different known foods (e.g., both on the receipt and known to be available), and the food score for the past time period, a shopping recommendation for the upcoming time period and the next shopping event. For example, the system may make recommendations on alternative purchases to replace items represented in the receipt data with personally healthier items based on the individualized needs of the participant (e.g., the participant data, such as the microbiome data and the like). In this manner, the system may gradually improve the pantry score and the health food score for the subsequent time periods. In some cases, the system may limit the number of shopping recommendations introduced per time period to reduce shock or resistance from the participant in changing their shopping habits.

In some cases, the shopping recommendations may be generated by one or more machine learned models trained on shopping behavior changes over time recorded by the system with respect to other participants. In some cases, the training data may include assigned weights to various recommendations based on historical effectiveness or adoption rates by other users or participants of the system in response to the same or similar recommendations. In some cases, the training data may include biases based on an assigned rating for a length of time that historical participants maintained the change in shopping behavior after a recommendation by the system. The one or more machine learned models trained may also be trained to provide shopping recommendations based on the personalized participant data (e.g., including but not limited to taste profiles, personal participant goals, health data, demographic data, microbiome data, and the like), food data personalized for the participants on each food, and health professional data, and the like.

In some implementations, the system may also generate a list of recommended meals or recipes for the subsequent or upcoming period of time. For example, the system may generate, for each meal of the upcoming time period, two or more recipes or meal plan options for the participant and/or group that the participant or group may select to form a shopping recommendation or list. In some cases, each participant may also include a list of preferred items and a list of excluded items that the system may utilize when generating the meal options. For example, the system may utilize the current pantry items in addition to known available items (e.g., such as in season items, regional available items, and the like with regards to the preferred item list and the excluded item list) when generating the meal or recipe options for each meal within the period of time.

In some cases, the participant or group may set budgets for each time period, limit a number of repeat ingredients over the period of time, and/or select main and/or secondary ingredients to include in the recommended meals or recipes. For example, the participant using the application hosted on the user device may add a maximum budget and a preferred budget threshold as well as a number of preferred proteins for the week (such as at least one meal including chicken and two meals including fish). In some case, the system may limit the number of main ingredients and/or secondary ingredients that a participant may select to ensure positive improvement on the health food score for the time period.

In some case, the system may be configured to access or communicate with third party grocery delivery systems to place orders for the shopping recommendations or list on behalf of the participant and/or group. In this manner, the participant may complete their shopping list and shopping event via the system without having to venture to the store thereby saving time and resources associated with travel.

Once the meal plan options are presented to the participants for the time period, the participant may select one meal or recipe option for each meal. In some cases, the participant may have selectable options on the user interface to request another alternative recipe as a replacement, change or substitute one or more ingredients of the currently presented recipe options, or the like. In some cases, when a group includes two or more participants, the system may allow for the participants to set meal selection rules for the time periods. For example, the system may allow each participant of the group to select one meal or recipe option in a round robin or other order to ensure each participant has meals that they will enjoy.

In this example, the system may send notifications or alerts to each user device associated with the participants when it is their turn to select a meal option. In other cases, the system may include thresholds meal limits (e.g., a number of meals each participant may select for the given time period) that may differ for each participant (e.g., one participant may select 10 meals while a second participant may only select 5 meals and the like). In other cases, the system may allow for vetoes of meal options selected by one or more other participant of a group or household to prevent any meals that one member of the group strongly opposes.

In some particular implementations, the system may include a meal plan or recipe generator. For example, the system may include a user interface with a text-based user data entry option and/or lists of ingredients that may be selected by the participant. In some cases, the participant may enter using generally conversational inputs including but not limited to descriptive sentences, sentence fragments, or items that may be used to generate a meal or recipe option. For example, the participants may enter inputs such as “I would like a meal including chicken and rice with Mediterranean spices that may be prepared a night in advance” and in response receive from the system one or more meal or recipe options.

In this example the system may utilize the user input into the text-based user data entry option as well as the participant data, historical consumption data (e.g., past meals), meal data of other meal options selected during a period of time, pantry data, food scores for various different foods personalized for the participant, any data associated with known group members, and the like. As discussed above, the system may utilize one or more machine learned models to generate the meal or recipe recommendations in the manner of recommending time period based meal planning.

For instance, the meal plan option or recipe recommendation machine learned models may include weights that are applied in response to a food score for a particular ingredient meeting or exceeding one or more threshold, an ingredient being considered a gut booster for the microbiome of the participant, food preferences and excluded items of the participant, allergies of the participant, time of year or day, personalized goals of the participant, known available pantry ingredients, resulting meal score for the participant, input by any third party supporting the participant (such as health professional, dietician or the like), and the like.

In some cases, once the meal or recipe options are presented on the display of the user device, the user may provide conversional text-based updates or edit requests to the presented options. In this manner, the system may update or modify original recommendations for the participant as an interactive meal or recipe generating session. In some cases, the system may utilize the participants entries via the text-based user data entry option to generate the time period based meal plan, as discussed herein.

In one specific example, the system may include a boost my meal or improve my meal selectable option for each meal or recipe provided. In this manner, the participant may choose to further improve or boost their health food score for the given time period one meal at a time. For example, in response to a selection of the improve my meal selectable option, the system may generate an updated version of the same recipe by swapping out ingredients in a contextually meaningful way.

For instance, the system may avoid changing the recipe (e.g., swapping a sandwich for a salad and the like). In some cases, the system may maintain one or more lists of substitute ingredients of each known or available ingredient. In some cases, the list of substitute ingredients may be weighted or ranked (e.g., personalized) based at least in part on the participant data of the participant. In this manner, the substitute ingredient list for each ingredient is customized for the individual participant. The system may swap the ingredients based on the individualized food score for different foods for the participant as well as known health benefits of the included ingredient when compared with the replaced ingredient.

In the cases of full time period meal planning by the system, the system may be configured to log the consumption data for the participant for the time period as the system is aware of the meals or recipes being consumed during the period of time. In this manner, the participant no longer has to manually enter the food consumed for each time period, unlike conventional food tracking system. Accordingly, the system discussed herein reduces time and computational resource consumption associated with manually logging consumption data for one or more given periods of time.

In some implementations, the system may include a coaching or target recommending engine for each participant. For example, at the expiration of each period of time, the system may generate one or more targets for a subsequent or next period of time. As an illustrative example, the system may provide recommended targets of “increasing fiber intake by 30 grams” or “reduce saturated fat consumption by 30 calories” over the next period of time. In some cases, the system may generate the target based on performance within the prior period of time (e.g., success or progress towards completing the prior target, the adherence to the meal plan, the participant data including participants targets, and the like).

In some case, the system may include one or more machine learned models for generating the time period based target for each participant. For example, the one or more machine learned models may be trained using historical data associated with other participant performance targets and progress and/or success of targets over various periods of time (e.g., day, week, month, year, or the like). In some cases, the system may set a primary target for a longer period of time (such as a month) and intermediate targets for sub-portions of the period of time (such as weekly or daily). In some cases, the intermediate targets may progress the participant towards completion of the primary target. In some implementations, the targets may be interactive, such that the primary target may adjust based on the performance of intermediate targets. For example, if a particular participant is exceeding the intermediate targets, the system may increase the primary target, introduce additional primary targets or intermediate targets, and the like to achieve improved results over a shorter period of time.

In the above example, the target for a time period is metric based (e.g., replace 30 calories of animal based protein with plant based protein” and the like). In other examples, the system may also include actions that may be performed that may move the participant towards achieving the target. In some cases, the actions may be less metric based. For example, an action may include “eat less red meat” or the like.

In some cases, the system may provide notifications, reminders, and/or alerts to each participant on progress towards completion of a target during the period of time. In some cases, the notifications may provide positive feedback and/or encouragement to assist the participant in progressing towards completion of the target. In other cases, the notifications may include educational material (e.g., blogs, podcasts, white papers, and the like) to provide additional data for the participant with respect to the particular target. In other cases, the notification may include recommended actions (e.g., “eat more beans”) to help the participant achieve the particular target (e.g. “increase fiber intake”). The notifications may also include or link to progress tracking indicators (such as visual indicators) of the participant's progress toward an intermediate and/or primary targets.

As described herein, various machine learned models or sets of models may be utilized by the system. In various examples, the sets of machine learned models may be the same or part of the same set or different sets to produce different results or outputs. The machine learned models may be generated using various machine learning techniques. For example, the models may be generated using one or more neural network(s). A neural network may be a biologically inspired algorithm or technique which passes input data (e.g., image and sensor data captured by the IoT computing devices) through a series of connected layers to produce an output or learned inference. Each layer in a neural network can also comprise another neural network or can comprise any number of layers (whether convolutional or not). As can be understood in the context of this disclosure, a neural network can utilize machine learning, which can refer to a broad class of such techniques in which an output is generated based on learned parameters.

As an illustrative example, one or more neural network(s) may generate any number of learned inferences or heads from the captured sensor and/or image data. In some cases, the neural network may be a trained network architecture that is end-to-end. In one example, the machine learned models may include segmenting and/or classifying extracted deep convolutional features of the sensor and/or image data into semantic data. In some cases, appropriate truth outputs of the model in the form of semantic per-pixel classifications (e.g., vehicle identifier, container identifier, driver identifier, and the like).

Although discussed in the context of neural networks, any type of machine learning can be used consistent with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like. In some cases, the system may also apply Gaussian blurs, Bayes Functions, color analyzing or processing techniques and/or a combination thereof.

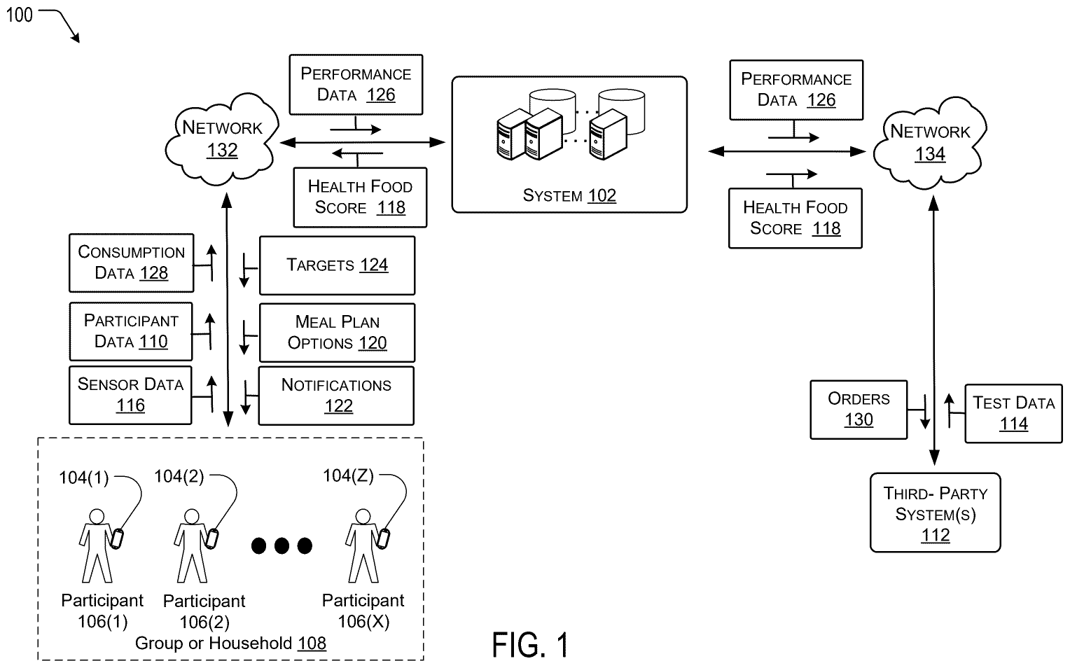

FIG. 1 is an example block diagram 100 of a system 102 for providing personalized and group health monitoring and meal planning, according to some implementations. As discussed above, the system 102 may be in communication with an application hosted on user devices 104(1)-(Z) for each participant 106(1)-(X). In the current example, the participant 106(1)-(X) are illustrated as part of a single group 108, however, it should be understood that the system 102 may being communication with one or more other participants that are associated with other groups or are participating with the system 102 as individuals.

In the current example, the system 102 is configured to encourage gradual health improvement and eating habits for each of the participants 106 and/or the group 108. For example, the system 102 may include an application hosted on each of a user devices 104 that may enable each participant 106 to engage with a participant specific account with the system 102 and via a joint group account with the system 102. In some cases, each participant 106 may provide participant data 110 (such as via an account initialization process or as part of regular updates) to the cloud-based system 102.

In various examples, the participant data 110 may include microbiome data, such as microbiome data identifying the presence and/or quantity of various bacteria and the like in a gut of the participants 106. For instance, the participant data 110 for each participant 106 may include lab generated data, such as when a participant 106 provides biological samples to a lab for testing. For example, each participant 106 may provide a lab with a sample, such as a stool sample, for microbiome analysis. As an example, metagenomic testing may be performed using the sample to allow the DNA of a microbiome of an individual to be digitalized. Generally, a microbiome analysis includes determining the composition and function of a community of microorganisms in a particular location, such as within the gut of each participant 106.

The participant data 110 may also include health data, blood data, glucose data, ketone data, nutrition data, genetic data, saliva data, biometric data, questionnaire data, psychological data (e.g., hunger, sleep quality, mood, and the like) as well as other types of data associated with each of the participants 106. Generally, health data may refer to any psychological, subjective and/or objective data that relates to and is associated with one or more individuals. The health data might be obtained through testing, self-reporting (such as via the application hosted on the user device), and the like.

In some examples, the health data includes wearable data obtained from technology worn and/or utilized by each participant 106. For instance, a participant 106 may wear a fitness device (included in a set of the user devices 104 associated with the participant 106 and registered with the account of the participant 106 at the system 102). As some examples, worn devices may include activity-monitoring devices that monitor motion, heart rate, sleep, calorie burn, activity, blood pressure, body temperature, and/or the like of the participant 106. As one specific example, worn devices may be a continuous glucose meter that monitors blood glucose levels often by measuring levels of glucose in interstitial fluid.

In some implementations, each participant 106 may also provide participant data 110 that may be utilized to predict the target values and/or changes to the target values and generate the nutritional recommendations using other devices such as blood glucose monitors discussed above, finger pricks which in some examples are used with dried blood spot cards, blood pressure monitors, and the like. Each participant 106 may also input data into one or more applications hosted on the user device 104. For example, a participant 106 may enter the foods the participant 106 consumed during a meal, how much the participant 106 slept, what exercise the participant 106 performed during a given period of time, how hungry the participant 106 are at one or more times of day including mealtimes, how the participant 106 feels, what medication the participant 106 consumed or consumes, and the like. The participant data 110 may also include data about their food preferences, medical guidance the participant 106 has received, or personalized food constraints/preferences, such as allergies, being vegan, gluten free, keto or other adhered diet, kosher, halal, or the like.

As another example, a participant 106 or a third-party 112 (such as a lab) may provide test data 114 determined from one or more tests, such as urinalysis test strips, blood test strips, and the like that may indicate a particular participant 106 and be included in the participant data 110 store data stored by the system 102. In some cases, the test data 114 may come from different sources, such as but not limited to from one or more of an individual, a lab, a doctor, an organization, and/or some other data source.

The system 102 may also receive, from the user device 104 associated with each participant 106 and/or the group 108, sensor data 116. The sensor data 116 may include data representative of food available to the system 102, such as for meal planning and health food score determining services. For example, the sensor data 116 may include image data of grocery shopping receipts, contents of a food storage area (e.g., a pantry, refrigerator, cold storage, freezer, or the like) and to provide the associated captured data to the cloud-based system. In some cases, the sensor data 116 may also include user input data representing the items or foods purchased by each participant 106 for a given period of time.

As discussed herein, the system 102 may be configured to determine nutritional data of each food item, such as the presence, type or class, quality, and the like of each food item represented in the sensor data 116. For example, the system 102 may determine a calorie count, fiber content, fat quantity and quality (e.g., monounsaturated or polyunsaturated, saturated, and the like), carbohydrate quantity and quality (e.g. glycemic index), vitamin content, mineral content, protein type (e.g., plant-based or animal-based) and content, salt/sodium content, water content, level of processing (e.g. NOVA) and the like associated with each food item.

In some implementation, the system 102 may generate a food score for each food item for each participant 106 and/or the group 108. For instance, the system 102 may generate the food score based at least in part on the nutritional data of the food item and the participant data 110. For example, the system 102 may generate personalized food scores for each food to participant 106 pair.

In some case, the system 102 may also generate a time period specific (such as daily, weekly, monthly, and the like) health food scores 118 for each of the participants 106 based at least in part on the participant data 110 associated with each participant 106 and the food data determined from the sensor data 116. For example, based on the food data representing a time periods food items (e.g., weekly) shopping receipt and the participant data 110, the system 102 may generate an individualized and personalized health food score 118 for each of the participants 106 and/or the group 108 representing the value of the purchased items (such as a proxy for consumed food items) for the individual participants 106. In some cases, the health food scores 118 may be based at least in part on the personalized food scores for each food and an assigned percentage, such as 25% for a group 108 having four participants 106. In some cases, the health food scores 118 may also be influenced or biased based on time of day consumed (e.g., as input by the participant 106 via the application hosted on the user device 104), season of the year, geographic location, weather, current health conditions (e.g., presence of a particular disease or sickness), activity level during the corresponding period, presence or absence of gut boosters or suppressors, and the like.

In some cases, in addition to the health food scores 118 for the given time periods, the system 102 may also generate one or more meal plan or recipe options 120 for the participants 106 and/or the group 108. In some examples, the system 102 may generate the meal plan options 120 for a given period of time or for a given meal. Each of the meal plan options 120 may be personalized for the participants 106 and/or group 108 based on the participant data 110 and the food data for each food item to participant 106 pair. In this manner, the system 102 may generate a meal plan options 120 that include an optimized meal plan score for each individual participant 106 given the available food items or ingredients.

In some cases, the system 102 may attempt to balance the meal plan options 120 to provide the highest health food scores 118 across the multiple participants 106 or group 108 (such as a highest average score, highest median score, highest summed score, or the like). In some cases, the system 102 may apply one or more thresholds for meal plan option 106 to ensure each participant 106 in a group 108 maintains a time period based health food score 118 or meal score equal to or exceeding the desired score.

In some implementations, the system 102 may also generate a list of recommended meals or recipes for a given period of time. For example, the system 102 may generate, for each meal of the given time period of time, two or more recipes or options 120 for the participants 106 and/or group. In some cases, each participant 106 may also include a list of preferred items and a list of excluded items that the system 102 may utilize when generating the meal plan options 120. For example, the system 102 may utilize the current pantry items in addition to known available items (e.g., such as in season items, regional available items, and the like with regards to the preferred item list and the excluded item list) when generating the meal plan 120. In some cases, the participants 106 and/or the group 108 may set budgets for the given period of time and/or select main and/or secondary ingredients to include in the recommended meals plan options 120. For example, each participant 106 using the application hosted on the user device 104 may set a maximum budget and a preferred budget threshold as well as a number of preferred ingredients. In some case, the system 102 may provide a list of main ingredients and a list of secondary ingredients that each participant 106 may select to ensure positive improvement on the health food score 118 for the given period of time.

In some cases, once the meal or recipe options 120 are presented to a participant 106 for the given period of time, the participant 106 may select one meal plan option 120 for each meal. In some cases, the participant 106 may have selectable options on the user interface to request another alternative option 120 as a replacement, to change or substitute one or more ingredients of the currently presented options 120, or the like.

In some cases, when a group 108 includes two or more participants 106 (such as in the illustrated example), the system 102 may allow for each participant 106 to apply meal selection rules for the given period of time. For example, the system 102 may allow each participant 106 of the group 108 to select one meal plan option 120 in a round robin or other order to ensure each participant 106 has meals that they will personally enjoy. In this example, the system 102 may send notifications 122 to each user device 104 associated with the participants 106 when it is the participant's 106 turn to select a meal plan option 120. In other cases, the system 102 may include thresholds meal limits (e.g., a number of meals each participant 106 may select for the given period of time). In other cases, the system 102 may allow for vetoes of meal plan options 120 selected by one or more other participant 106 of a group 108 to prevent any meals that one participant 106 of the group 108 strongly opposes.

In some particular implementations, the system 102 may include a user interface with a text-based user data entry option and/or lists of ingredients that may be selected by a participant 106. In some cases, the participant 106 may enter using generally conversational inputs including but not limited to descriptive sentences, sentence fragments, or items that may be used to generate a meal plan option 120. For example, the participants may enter inputs such as “I would like a meal including lentils and beans with moderate spice levels that has 30 minutes or less prep time.” In response the system 102 may generate one or more meal plan options 120 that complies with the input.

In this example the system 102 may utilize the user input into the text-based user data entry option as well as the participant data 110 for each member of a group 108, historical consumption data (e.g., past meals), meal data of other meal plan options 120 selected during the given period of time, pantry data, food scores for various different foods personalized for the participants 106, any data associated with known members of the group 108, and the like.

In some cases, once the meal plan options 120 are presented on the display of the user device 104, if the participant 106 is not fully satisfied with the options 120, the participant 106 may provide conversional text-based updates or edits request to the presented options 120 and the system 102 may generate additional meal plan options 120. In this manner, the system 102 may update or modify original recommendations for the participant 106 as an interactive meal or recipe generating session.

In one example, the system 102 may include a boost my meal or improve my meal selectable option for each meal plan option presented. In this manner, each participant 106 may choose to further improve or boost their health food score 118 for the given period of time on a meal by meal basis. For example, in response to a selection of the improve my meal selectable option, the system 102 may generate an updated version of the same recipe by swapping out ingredients in a contextually meaningful way. For instance, the system 102 may avoid changing the recipe (e.g., swapping a stew for a pasta and the like).

In some cases, the system 102 may maintain one or more lists of substitute ingredients of each known or available ingredient. In some cases, the list of substitute ingredients may be weighted or ranked (e.g., personalized) based at least in part on the participant data 110 of each participant 106. In this manner, the substitute ingredient list for each ingredient is customized for the individual participant 106. In some cases, the system 102 may swap the ingredients based on the individualized food score for different food items used as ingredients for the participant 102 as well as known health benefits of the included ingredient when compared with the replaced ingredient.

In some implementations, the system 102 may include a coaching or target recommending engine for each participant 106. For example, at the expiration of the given period of time (e.g., the week also associated with the meal planning discussed above and/or a different period of time), the system 102 may generate one or more targets 124 for the subsequent period of time.

As an illustrative example, the system 102 may provide recommended targets 124 such as “increasing fiber intake by 30 grams” or “reduce saturated fat consumption by 30 calories” over a given period of time. In some cases, the system 102 may generate a target 124 based on performance data 126 associated with the prior period of time (e.g., success or progress towards completing the prior target 124, the adherence to the meal plan, the participant data 110, and the like).

In some cases, the system 102 may set a primary target 124 for a longer period of time (such as a month) and intermediate target 124 for sub-portions of the longer period of time (such as weekly or daily). In some cases, the intermediate target 124 may progress the participant 106 towards completion of the primary target 124. In some implementations, the target 124 may be interactive, such that the primary target 124 may adjust based on the performance of intermediate target 124. For example, if a particular participant 106 is exceeding the intermediate target 124, the system 102 may increase the primary target 124, introduce additional primary targets 124 or intermediate targets 124, and the like to achieve improved results over a shorter period of time.

In the above example, the target 124 for a time period is metric based (e.g., replace 30 calories of animal based protein with plant based protein” and the like). In other examples, the target 124 may be less metric based. For example, a target may include “avoid eating between meals” or the like.

In addition to health goals of the participants 106 and the targets 124, the system may also generate and provide actions (such as via the notification 122) that would facilitate progress towards completion of the target 124 by each participant 124. For example, the actions may include eating based recommendations such as eat more beans to increase fiber or eat less red meat to reduce fat consumption.

In some cases, the system 102 may provide notifications 122 (e.g., reminders and/or alerts) to each participant 106 on progress towards completion of a target 124 during the given period of time. In some cases, the notifications may provide positive feedback and/or encouragement to assist each participant 106 in progressing towards completion of the target 124. In other cases, the notifications 122 may include educational material (e.g., blogs, podcasts, white papers, and the like) to provide additional data for each participant 106 with respect to a particular target 124. In other cases, the notifications 122 may include recommended actions (e.g., “eat more beans”) to help the participant 106 achieve the particular target 124 (e.g. “increase fiber intake”). The notifications 122 may also include or link to progress tracking indicators (such as visual indicators) of the participant's progress toward an intermediate and/or primary target 124.

In some cases, the system 102 may be configured to log the consumption data 128 on behalf each participant 106 for each given period of time, as the system 102 is aware of the meals or recipes being consumed (e.g., via the sensor data 116 and/or meal plan options 120). In this manner, each participant 106 no longer has to manually enter the food items consumed for each period of time, unlike conventional food tracking systems. Accordingly, the system 102 discussed herein reduces time and computational resources consumption associated with manually logging consumption data for one or more given periods of time. Additionally, the logging by the system 102 discussed herein reduces error caused by human data entry, reduces time investment of participants 106 (often resulting in greater health benefits and participation by the users), increases flexibility in meal planning, as well as providing each participant 106 with more personalized health results, and the like.

In some case, such as the current example, the system 102 may be configured to access or communicate with third-party systems 112 (such as a third-party grocery delivery service) to place orders 130 on behalf of each participant 106 and/or group 108. The orders 130 may be generated based on known food items in a food storage area and/or the meal plan options 120 selected by each participant 106 of a group 108 for the corresponding given period of time. In this manner, each participant 106 may complete their shopping list and shopping events via the system 102 without having to venture to the store thereby saving time and resources associated with travel.

In the current example, the system 102, upon completion of a given period of time, may generate the performance data 126 based on the meal plan options 120 selected by the user and any sensor data 116 (e.g., receipt data, wearable data, user input data, and the like) received from the user device 104. The performance data 126 may include the health food score 118 for the corresponding week as well as any progress made toward completion of the targets 124. The system 102 may provide the performance data 126 to the user device 104 for review by each participant 106 as well as to third-party systems 112, such a health care professional or dietician who is supporting the corresponding participant 106 and is authorized by the corresponding participant 106 to receive the performance data 126.

In the current example, the data may be transmitted between various systems and user devices using networks, generally indicated by 132-134. The networks 132-134 may be any type of network that facilitates communication between one or more systems and may include one or more cellular networks, radio, WiFi networks, short-range or near-field networks, infrared signals, local area networks, wide area networks, the internet, and so forth. In the current example, each network 132-134 is shown as a separate network but it should be understood that two or more of the networks may be combined or the same.

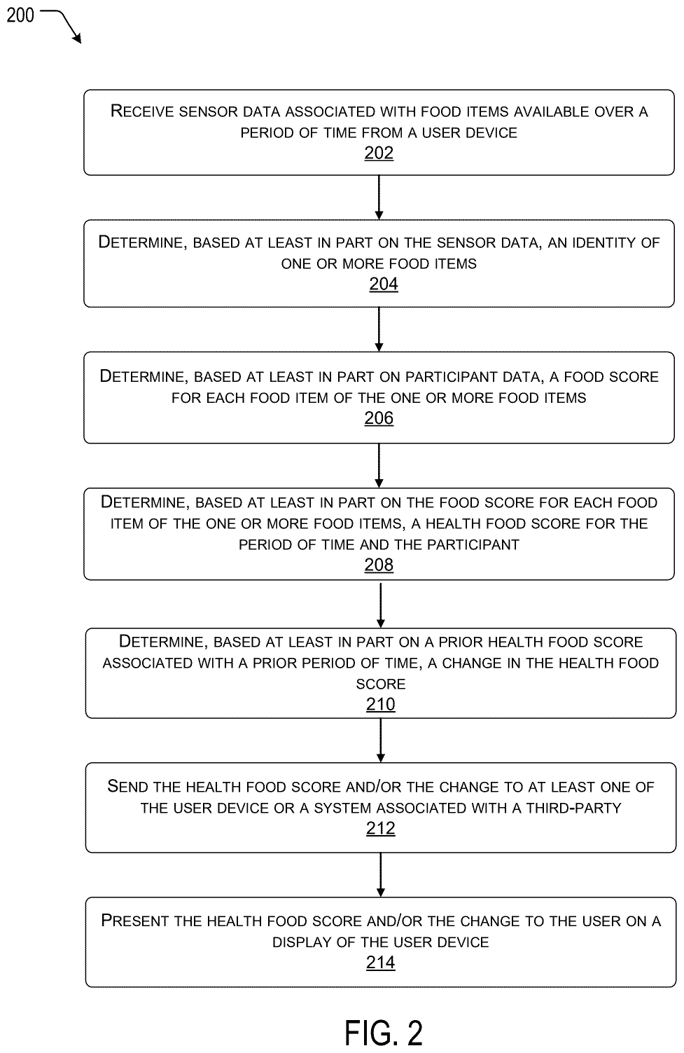

FIGS. 2-5 are flow diagrams illustrating example processes associated with the system discussed herein. The processes are illustrated as a collection of blocks in a logical flow diagram, which represent a sequence of operations, some or all of which can be implemented in hardware, software, or a combination thereof. In the context of software, the blocks represent computer-executable instructions stored on one or more computer-readable media that, when executed by one or more processor(s), perform the recited operations. Generally, computer-executable instructions include routines, programs, objects, components, encryption, deciphering, compressing, recording, data structures and the like that perform particular functions or implement particular abstract data types.

The order in which the operations are described should not be construed as a limitation. Any number of the described blocks can be combined in any order and/or in parallel to implement the processes, or alternative processes, and not all of the blocks need be executed. For discussion purposes, the processes herein are described with reference to the frameworks, architectures and environments described in the examples herein, although the processes may be implemented in a wide variety of other frameworks, architectures or environments.

FIG. 2 is a flow diagram illustrating an example process 200 associated with the system 102 for providing personalized and group health monitoring and meal planning of FIG. 1, according to some implementations. As discussed herein, a system, such as a cloud-based system in communication with one or more user devices for each participant, may generate health food scores for a participant based at least in part on receipt data representing food items purchased by the participant for a given period of time.

At 202, the system may receive sensor data associated with food items available over a period of time from a user device. For example, the system may receive sensor data, such as an image of a receipt for the weekly shopping event of the participant, from a user device. In other cases, the sensor data may be an image of a food storage area, such as a pantry, refrigerator, freezer, and the like, or a combination there.

At 204, the system may determine, based at least in part on the sensor data, an identity of one or more food items. For example, using one or more machine learned models and/or other software techniques the system may segment, classify, and extra data from each of line of a receipt represented in the sensor data.

At 206, the system may determine, based at least in part on participant data, a food score for each food item of the one or more food items. For example, the system may utilize the participant data and/or nutritional data known about each food item to generate a food score for each item. In some cases, the system may detect new food items that have not been previously assigned a food score and only generate food scores for those new items. In this manner, the system may build a personalized database with food scores for food items as the participant consumes them for the individual participant.

At 208, the system may determine, based at least in part on the food score for each food item of the one or more food items, a health food score for the period of time and the participant. For example, using the food items represented by the sensor data and the food score for each item, the system may generate a health food score for the participant. In this case, the health food score may represent the value of the food items if consumed by the participant during the given period of time. In some cases, the participant may be able to select food items that were actually consumed during the given period of time to improve the quality of the health food score generated by the system.

At 210, the system may determine, based at least in part on a prior health food score associated with a prior period of time, a change in the health food score. For example, the system may compare the prior health food score with the newly generated health food score to determine a change either positive or negative that is personalized for the individual participant. In some cases, the system may utilize the change data to determine progress or overall improvement or decline in health by the participants food choices. In some cases, the system may utilize the change data to generate a notification for the participant including educational information and/or health benefits caused by the change in health food score from period of time to period of time.

At 212, the system may send the health food score and/or the change to at least one of the user device or a system associated with a third-party and, at 214, the system may present the health food score and/or the change to the user on a display of the user device. For example, the system may send the health food score and/or the change data to the user device for presenting on a display to a participant, such that the participant can monitor their food consumption and associated health benefit from week to week. In some cases, the system may also send the health food score and/or the change data to a third-party system such as a health professional (potentially treating one or more condition) and/or a dietician supporting the participant so that the third-party may also monitor the participant's food consumption and associated health benefit from week to week.

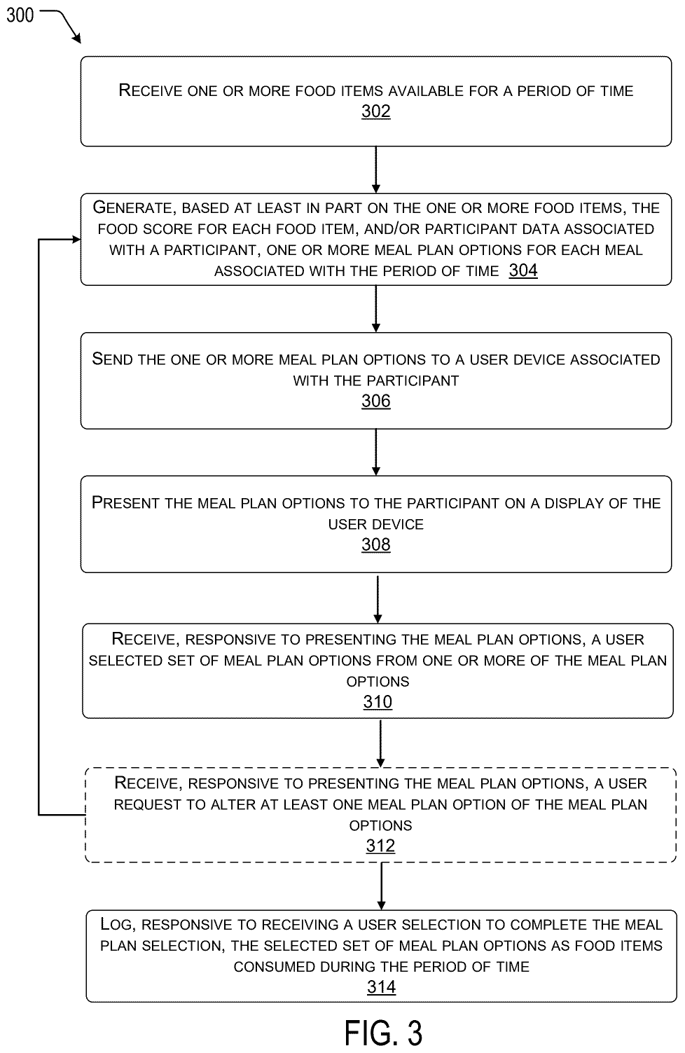

FIG. 3 is a flow diagram illustrating an example process 300 associated with the system 102 for providing personalized and group health monitoring and meal planning of FIG. 1, according to some implementations. As discussed herein, a system may be configured to generate meal plan options for participants over a given period of time such as a week, two weeks, a month, or the like. In some cases, the system may also log consumption data representing the food items consumed by the participant based at least in part on the meal plan options selected during the period of time.

At 302, the system may receive one or more food items available for a period of time. For example, the system may receive sensor data, such as image data of a food storage area and/or a receipt associated with a shopping event performed by the participant. In some cases, the system may store the available food item data and update the available food item data as items are consumed and/or purchased.

At 304, the system may generate, based at least in part on the one or more food items, the food score for each food item, and/or participant data associated with a participant, one or more meal plan options for each meal associated with the period of time. For example, the system may generate two recipes or options for each meal that the participant normally consumes over the period of time. In some cases, the participant may select which meals on which weekdays the participant typically consumes. As an illustrative example, the participant may indicate they consume breakfast and dinner each day of the week but lunch only on Saturday. Similarly, the participant may indicate that on Wednesdays, they work late and consume a third meal after dinner. The system may then generate one or more meal plan options for the participant for each meal indicated during the given period of time.

At 306, the system may send the one or more meal plan options to a user device associated with the participant and, at 308, the system may present the meal plan options to the participant on a display of the user device. In some case, the system may provide a health food score for each meal option presented. The health food score may be customized for the individual participant. In some case, when the participant is associated with a group, the system may present a health food score for each participant that will partake in the associated meal.

At 310, the system may receive, responsive to presenting the meal plan options, a user selected set of meal plan options from one or more of the meal plan options. For example, the participant or participants of the group may select meal plans for each available meal during the given period of time. As discussed above, the system may allow for a rotation in the selection, thresholds for each participant of the group to select a minimum or maximum number of meals or a ratio of selection options per participant (e.g., a first participant selects 3 meals for every one meal selected by a second participant), and the like.

At 312, the system may also optionally receive, responsive to presenting the meal plan options, a user request to alter at least one meal plan option of the meal plan options. For example, the participant may not be in the mood for chicken and request that a particular meal plan option be changed to include a different protein source or the like. If the request is received, the process 300 may return to 304 with respect to each meal that a request was made and the system may generate one or more additional meal plan options for these meals.

At 314, the system may log or record, responsive to receiving a user selection to complete the meal plan selection, the selected set of meal plan options as food items consumed during the period of time. For example, the system may log the consumption of each meal upon the mealtime passing. In some cases, the system may apply a percentage or scaling factor to each meal plan option as the option is logged based on an amount of food consumed or an amount of the meal consumed by each participant.

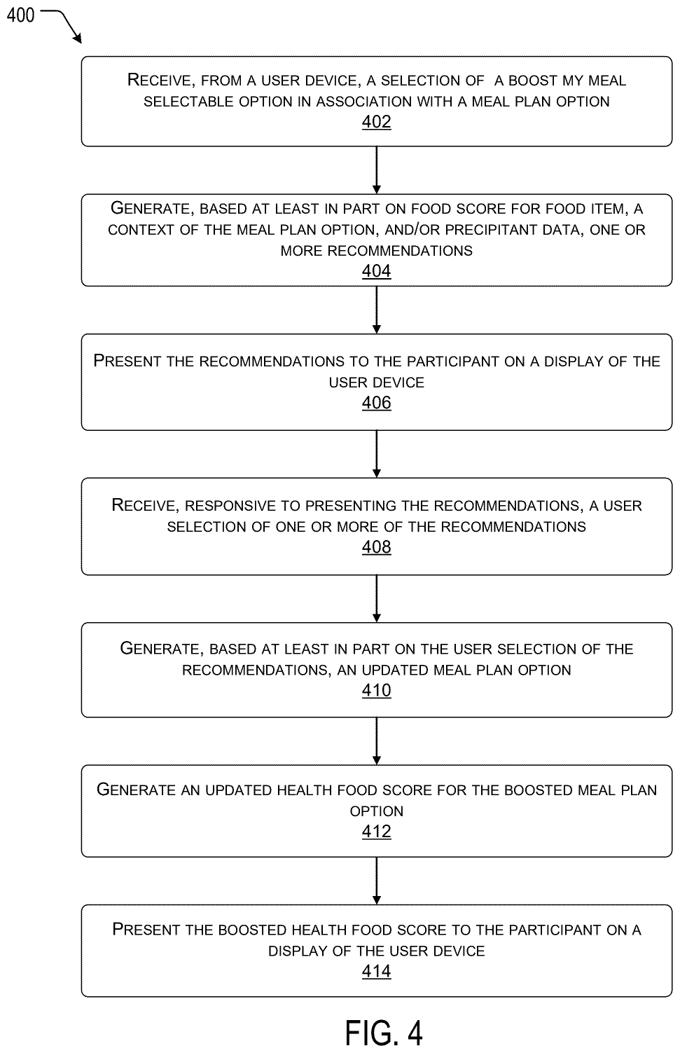

FIG. 4 is a flow diagram illustrating an example process 400 associated with the system 102 for providing personalized and group health monitoring and meal planning of FIG. 1, according to some implementations. As discussed herein, the system, such as a cloud-based system in communication with one or more user devices for each participant, may allow a participant to boost or otherwise improve the quality of each individual meal based on the participant's personalized health food score for that meal. As discussed here, the health food score for each meal is based on the participant data and the food score for the same participant of each ingredients (or food items) included in the meal. In this manner, the food score may be adjusted based on the participants individual microbiome, allergies, demographic information, physical location or residence (e.g., individuals living in the northern portions of North America may benefit more from vitamin D rich foods than individuals living in the southern portions of North America), medical conditions, or the like.

At 402, the system may receive, from a user device, a selection of a boost my meal selectable option in association with a meal plan option. For example, as part of the user interface, discussed in more detail below, may include an option to add a meal plan option to given period of time or a specific meal of a meal plan, to modify the meal plan option, and/or to boost the meal plan option.

At 404, the system may generate, based at least in part on a food score for a food item, a context of the meal plan option, and/or precipitant data, one or more recommendations to boost the specified meal. For example, the boost my meal plan option may provide one or more options to swap ingredients included in the meal plan option, to remove one or more ingredients, and/or add one or more ingredients to the meal plan option. In various cases, the system may also generate a recipe and nutritional data together with the health food score of the original meal plan option, the boosted meal plan option, and the resulting changes to provide an ease of comparison between the original and boosted meal plan option.

At 406, the system may present the recommendations to the participant on a display of the user device. For example, if the system generates two or more boosted meal plan options, the two or more options may be presented on the display including the change list (e.g., ingredient swap, addition, or removal recommendation) and the resulting change in the health food score and/or other nutritional values.

At 408, the system may receive, responsive to presenting the recommendations, a user selection of one or more of the recommendations and, at 410 generate, based at least in part on the user selection of the recommendations, an updated meal plan option. For example, the participant may select a first recommendation to swap tilapia for salmon and a second recommendation to add olives to the meal plan option. In response, the system may generate a new meal plan option including the recipe, health food score personalized for the participant as discussed herein, and, for instance, update a grocery shopping list or order with a third-party system on behalf of the participant, and the like. In some cases, when generating the boosted meal plan option, the system may change one or more additional ingredients or food items to ensure that the meal is still enjoyable to consume (such as based at least in part on a participants taste preferences and profile).

At 412, the system may generate an updated health food score for the boosted meal plan option and, at 414, the system may present the boosted health food score to the participant on a display of the user device. For example, the system may generate the health food score and any changes between the original meal plan option and the boosted meal plan option including any intentional ingredient changes (e.g., those selected by the participant via the recommendations) and/or any incidental ingredient changes (e.g., changes or swaps to maintain a taste profile). In this manner, the health food score and/or change data may differ between those presented in the one or more recommendations and the final resulting boosted meal plan option. It should also be understood that a participant may boost the same meal multiple times or rounds to continue to improve the health food score. Accordingly, either following (e.g., via a subsequent display interface) or together with the boosted meal plan option, the system may present additional recommendations for further improving the meal plan option based on the personalized nutritional needs of the participant.

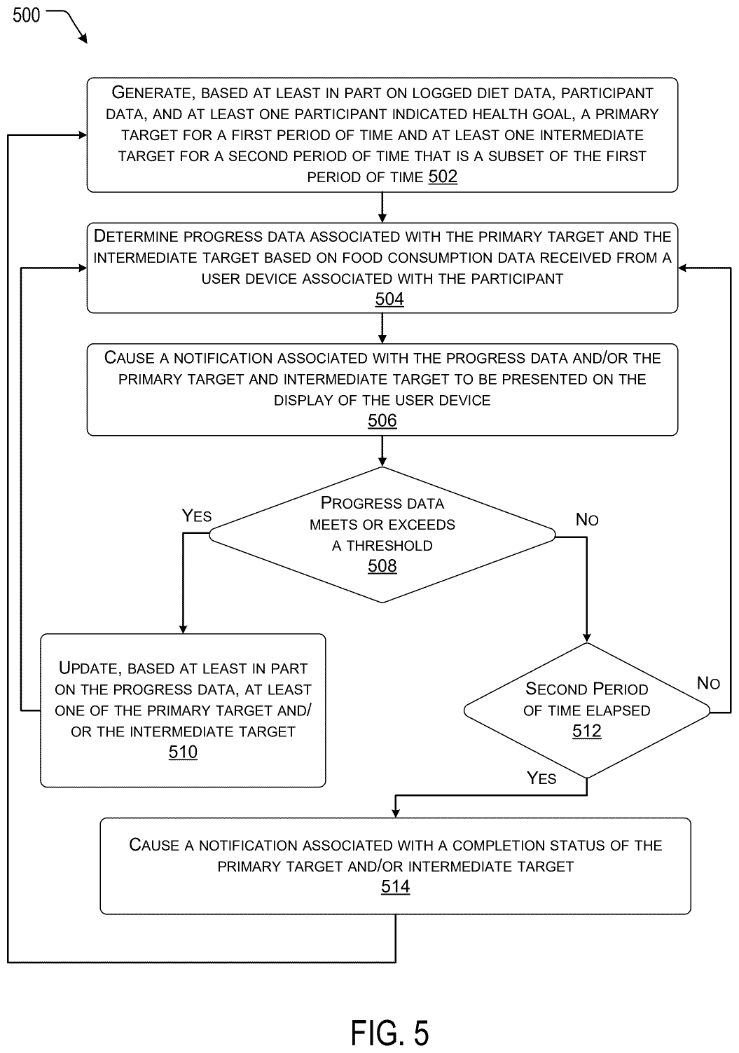

FIG. 5 is a flow diagram illustrating an example process 500 associated with the system 102 for providing personalized and group health monitoring and meal planning of FIG. 1, according to some implementations. As discussed herein, the system may be configured to provide personalized or individualized targets to encourage gradual healthier eating by each participant. In this manner, the system, the cloud-based system and the application hosted on the user device, may act as a virtual coach or trainer with respect to each participant's food habits.

At 502, the system may generate, based at least in part on logged diet or consumption data, participant data, and at least one participant indicated goal (e.g., an overall health goal provided by the user, a health care professional, a dietician, or other third-party), a primary target for a first period of time and at least one intermediate target for a second period of time that is a subset of the first period of time. In some implementations, the system may only generate a single target but in other implementations the system may generate two or more related targets, for example, the primary and intermediate targets discussed herein.

At 504, the system may determine progress data associated with the primary target and the intermediate target based on food consumption data received from a user device associated with the participant. For example, the system or the participant may log consumption data after each meal, the participant or a worn user device may provide activity data, and the like.

At 506, the system may cause a notification associated with the progress data and/or the primary target and intermediate target to be presented on the display of the user device. For example, the notification may be periodic or otherwise based on a time trigger. In other examples, the notifications may be provided at various thresholds or milestones between each target, such as 25%, 50%, and/or 75% completion. In some cases, the notifications may also include or provide access to educational material associated with the target, such as an article or podcast discussing the importance of eating more lean protein or the like. In some cases, the notifications may also include or provide access to recommended actions associated with the target, such as a recommendation to eat more beans to help achieve a target of increasing fiber intake.

In some examples, the system may also generate actions that be performed by a participant that may increase progress towards completion of a target (e.g., either primary or intermediate). The actions may include eating habits, health actions, and the like that the participant may perform. For example, the actions may include “eat more vegetables this week” or “walk in the morning before breakfast”.

At 508, the system may determine if the progress data meets or exceeds one or more thresholds. If the progress data indicates the progress is greater than or equal to the one or more thresholds, the process 500 may advance to 510. However, if the progress data does not meet or exceed the one or more thresholds, the system may progress to 512. In some cases, the one or thresholds may be the intermediate or primary target (e.g., the participant completed the target ahead of schedule).

At 510, the system may update, based at least in part on the progress data, at least one of the primary target and/or the intermediate target. For example, if the target was to consume three or more vegetarian meals, the system may update the target to consume one additional meal vegetarian meal during the given period of time or the like. Once, the target has been updated, the process 500 may return to 504.

At 512, the system may determine if the second period of time had elapsed. For example, the system may determine if the period of time associated with one or more intermediate targets and/or the primary target has elapsed. If the period of time has not elapsed, the process 500 may return to 504. Otherwise, the process 500 may advance to 514.

At 514, the system may cause a notification associated with a completion status of the primary target and/or intermediate target. For example, if the participant completed the corresponding target then the system may provide congratulations to the participant. However, if the participant did not achieve their corresponding target, the system may provide encouragement for the subsequent period of time, educational materials, recommended actions, and/or the like.

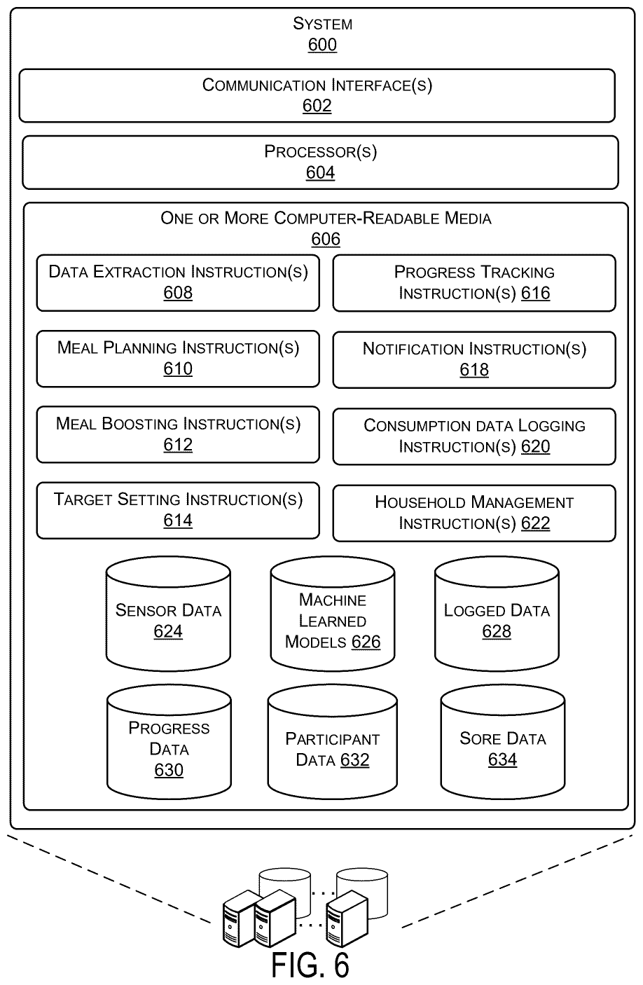

FIG. 6 is an example system 600 for providing personalized and group health monitoring and meal planning that may implement the techniques described herein according to some implementations. The system 600 can include one or more communication interface(s) 602 that enable communication between the system 600 and one or more user devices associated with one or more participants. For instance, the communication interface(s) 602 can facilitate communication with other proximate sensor systems and/or other facility systems. The communications interfaces(s) 602 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The system 600 may include one or more processors 604 and one or more computer-readable media 606. Each of the processors 604 may itself comprise one or more processors or processing cores. The computer-readable media 606 is illustrated as including memory/storage. The computer-readable media 606 may include volatile media (such as random access memory (RAM)) and/or nonvolatile media (such as read only memory (ROM), Flash memory, optical disks, magnetic disks, and so forth). The computer-readable media 606 may include fixed media (e.g., RAM, ROM, a fixed hard drive, and so on) as well as removable media (e.g., Flash memory, a removable hard drive, an optical disc, and so forth). The computer-readable media 606 may be configured in a variety of other ways as further described below.

Several modules such as instructions, data stores, and so forth may be stored within the computer-readable media 606 and configured to execute on the processors 604. For example, as illustrated, the computer-readable media 606 stores data extraction instructions 608 (e.g., extracting data from sensor data, user inputs, and the like), meal planning instructions 610 (e.g., to generate one or more meal plan options personalized for each participant), meal boosting instructions 612 (e.g., to make meal plan recommendations personalized for each participant or group), target setting instructions 614 (e.g., to assist in setting time period based targets personalized for each participant), progress tracking instructions 616 (e.g., to generate progress data 630 associated with one or more of the time period based targets), notification instruction 618 (e.g., to generate and transmit notifications personalized for each participant), consumption data logging instruction 620 (e.g., to log data associated with meal consumption and activity on behalf of each participant), group management instruction 622 (e.g., to assist with managing group members and supervising personalization of meals and targets between members) as well as other instructions, such as an operating system. The computer-readable media 606 may also be configured to store data, such as sensor data 624, machine learned models 626, logged data 628, progress data 630, participant data 632, and score data 634 as well as other data.

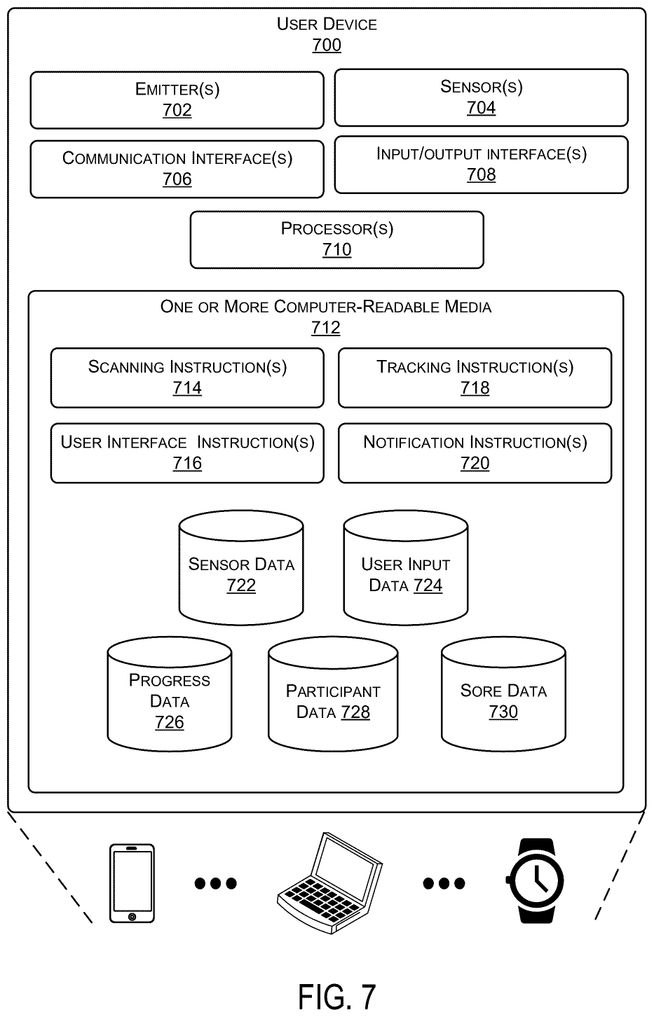

FIG. 7 is an example user device 700 that may implement the techniques described herein according to some implementations. In some cases, the user device 700 may be a hand-held electronic device equipped with sensors, a user interface, and one or more hosted applications. In some examples, the user device 700 may be implemented as a hand-held device in communication with a cloud-based system for providing personalized health food scores, as discussed herein.

In some examples, the user device 700 may include one or more emitters 702. The emitters 702 may be mounted on an exterior surface of the user device 700 in order to output illumination or light into a physical environment, body feature (e.g., a wrist in the case of a blood pressure monitor or the like), or the like. The emitters 702 may include, but are not limited to, visible lights emitters, infrared emitters, ultraviolet light emitters, LIDAR systems, and the like. In some cases, the emitters 702 may output light in predetermined patterns, varying wavelengths, or at various time intervals (e.g., such as pulsed light).

The user device 700 may also include one or more sensors 704. The sensor 704 may include image sensors, depth sensors, motion sensors, position sensors, health sensors, wearable sensors, body sensors, and the like. For example, the sensors 704 may include image devices, spectral sensors, IMUs, accelerometers, gyroscopes, depth sensors, infrared sensors, GPS systems, blood sugar sensor, and/or the like.

The user device 700 may also include one or more communication interfaces 706 configured to facilitate communication between one or more networks, one or more cloud-based system(s), and/or one or more mobile or user devices. In some cases, the communication interfaces 706 may be configured to send and receive data with, for instance, the cloud-based system as discussed above. The communications interfaces(s) 706 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

In the illustrated example, the user device 700 also includes an input and/or output interface 708, such as a projector, a virtual environment display, a traditional 2D display, buttons, knobs, and/or other input/output interfaces. For instance, in one example, the interfaces 708 may include a flat display surface, such as a touch screen configured to allow a user of the device 700 to consume content (such as scanning instructions, 3D models, and the like) and to provide feedback in the form of touch inputs.

The user device 700 may also include one or more processors 710, such as at least one or more access components, control logic circuits, central processing units, or processors, as well as one or more computer-readable media 712 to perform the function associated with the virtual environment. Additionally, each of the processors 710 may itself comprise one or more processors or processing cores.

Depending on the configuration, the computer-readable media 712 may be an example of tangible non-transitory computer storage media and may include volatile and nonvolatile memory and/or removable and non-removable media implemented in any type of technology for storage of information such as computer-readable instructions or modules, data structures, program modules or other data. Such computer-readable media may include, but is not limited to, RAM, ROM, EEPROM, flash memory or other computer-readable media technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, or any other medium that can be used to store information and which can be accessed by the processors 710.

Several modules such as instructions, data stores, and so forth may be stored within the computer-readable media 712 and configured to execute on the processors 710. For example, as illustrated, the computer-readable media 712 may scanning instructions 714, user interface instructions 716, tracking instructions 718, notification instructions 720, as well as other instructions. The computer-readable media 712 may also store data such as sensor data 722, user input data 724, progress data 726, participant data 728, score data 730, and the like.

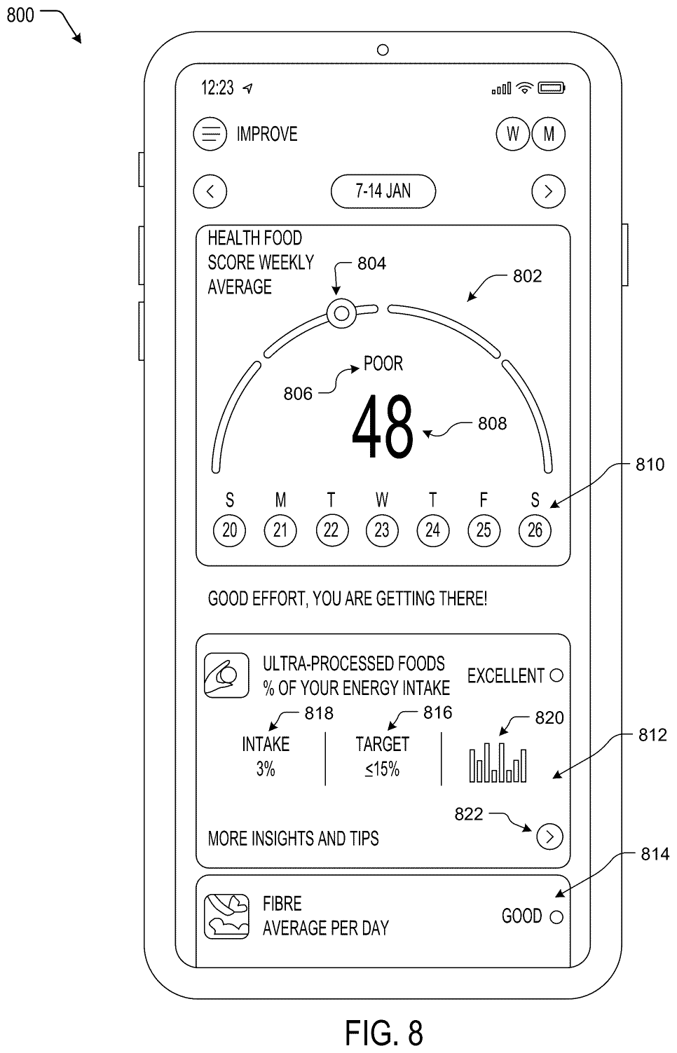

FIG. 8 is an example pictorial diagram illustrating an example user interface 800 associated with a personalized health food score 802 of a participant according to some implementations. For instance, in the current example, a user device, such as a smartphone, may display the user interface 800 presenting the health food score 802 for a given period of time (e.g., a week in the current example). In the current example, the user interface 800 may present the health food score 802 as a position along a slider 804, a text-based quality indicator 806, and a numerical value (such as out of 100) 808.

The user interface 800 may also include an indicator of the period of time 810, such as the selected days of the week. The current user interface 800 may also include one or more targets for the period of time either selected by the participant and/or generated by the system. For example, a first display area 812 associated with a first target and a second display area 814 associated with a second target are currently illustrated. It should be understood that the participant may adjust the user interface 800, such as by vertically scrolling the interface 800, to view additional information, such as the remainder of the second display area 814, on the display.

With regards to the first display area 812, the user interface 800 may present a target 816, progress data 818, and a graphical representation of progress data on various subsets of the period of time (currently daily) 820. The first display area 812 may also include a user selectable option 822 to receive advice or recommendations associated with achieving the target 816.

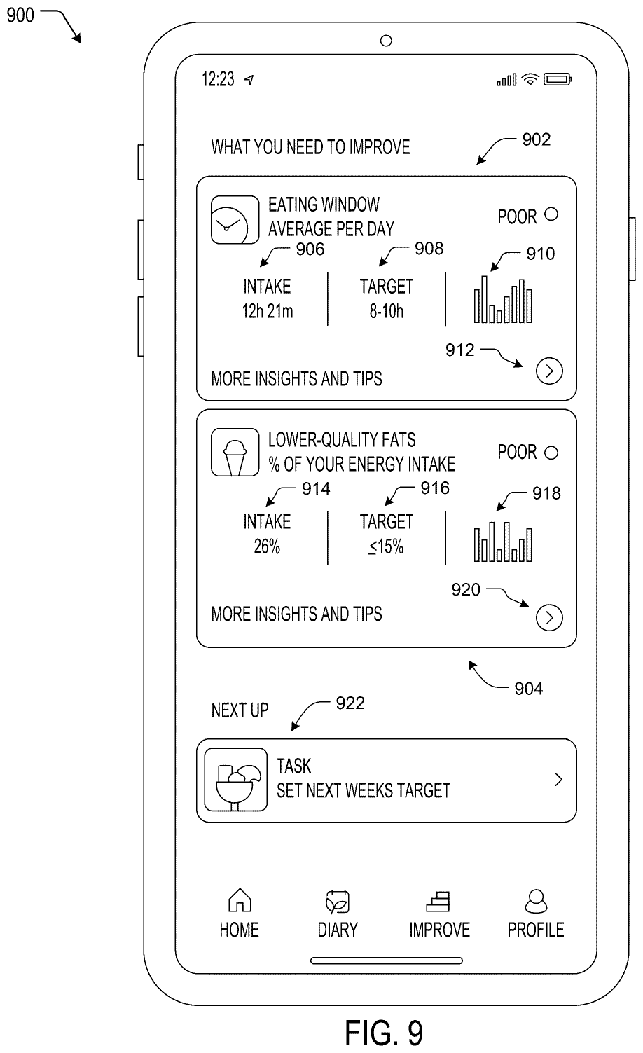

FIG. 9 is an example pictorial diagram illustrating an example user interface 900 associated with a personalized recommendations to improve the participant's health food score according to some implementations. In the current example, the user interface 900 may provide notifications or display areas associated with progress, achievement, and/or completion data or metrics associated with one or more targets. For instance, in the illustrated example, the user interface 900 may include a first display area 902 associated with a first target and a second display area 904 associated with a second target. As discussed above, for each of the display areas 902 and 904, the user interface 900 may display the target 908 and 916, the progress data or completion metric 906 and 914, and a graphical representation of progress data 910 and 918, respectively. The user interface 900 may also include a user selectable option 912 to receive advice or recommendations associated with achieving the target 908 and, likewise, a user selectable option 920 to receive advice or recommendations associated with achieving the target 916.

In the current example, the user interface 900 may also include a user selectable option 922 to review, manage, and determine targets for the upcoming period of time. For example, as the participant failed to complete either targets 908 or 916 in the current example, the next week targets may include similar targets.

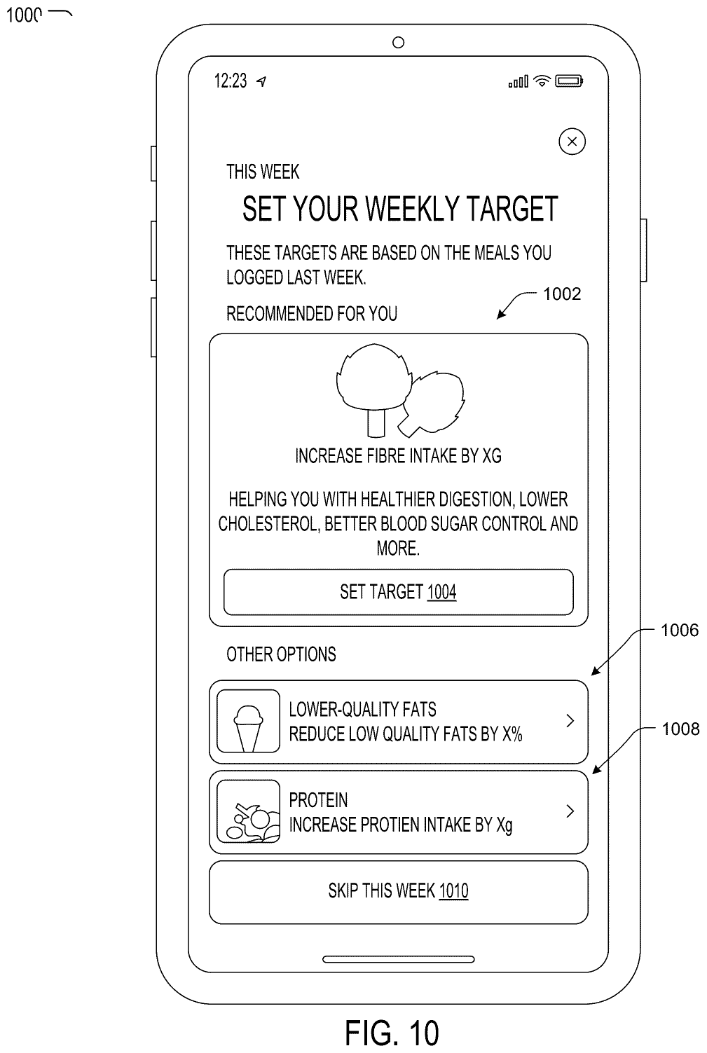

FIG. 10 is an example pictorial diagram illustrating an example user interface 1000 associated with setting a personalized target for a given period of time according to some implementations. In the current example, the user interface 1000 may be utilized to set a target for a given period of time, as discussed herein. As shown, the system may recommend a target 1002 for the upcoming or current period of time (such as a week). The recommended target 1002 may be selected by the participant via a user selectable option 1004.

If, for instance, the participant would prefer different targets than the target 1002, the user interface 1000 may also present additional target options, as illustrated by 1006 and 1008. In some cases, the user interface 1000 may also allow the participant to skip a period of time, such as via illustrated elective option 1010.

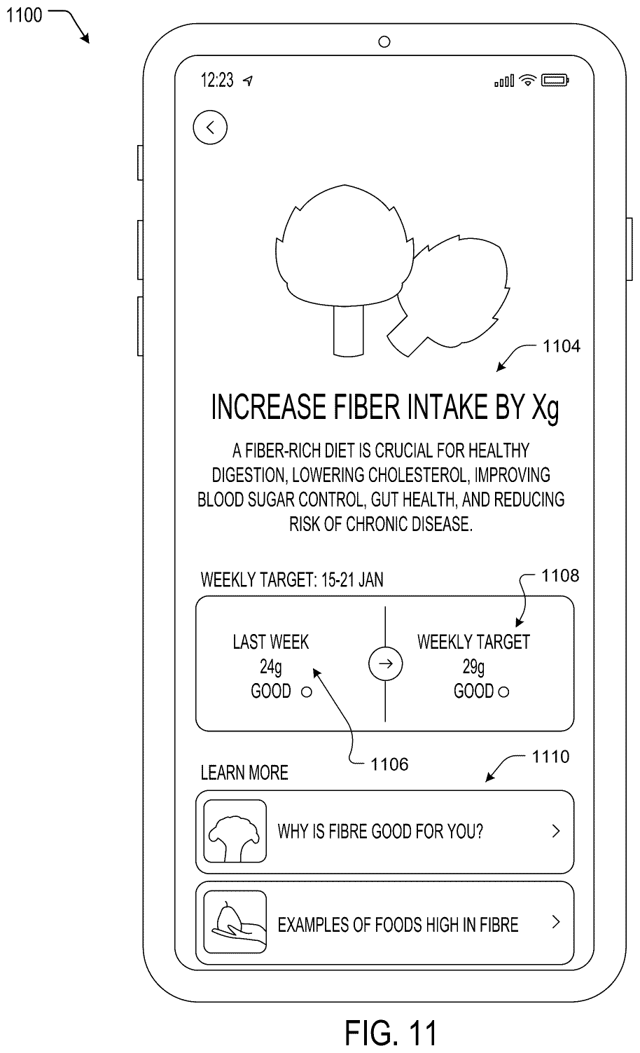

FIG. 11 is another example pictorial diagram illustrating an example user interface 1100 associated with setting a personalized target for a given period of time according to some implementations. In the current example, the participant may review a potential target 1104 for the upcoming and/or current period of time. For instance, the potential target 1104 may be to increase fiber by a number of grams. In setting the number of grams, the user interface 1102 may provide data on last periods intake 1106 (either as a prior target or as indicated by the consumption data for the prior period) and a recommended target 1108. The user interface 1100 may also provide user selectable links or options 1110 to provide additional information related to the recommended target 1108.

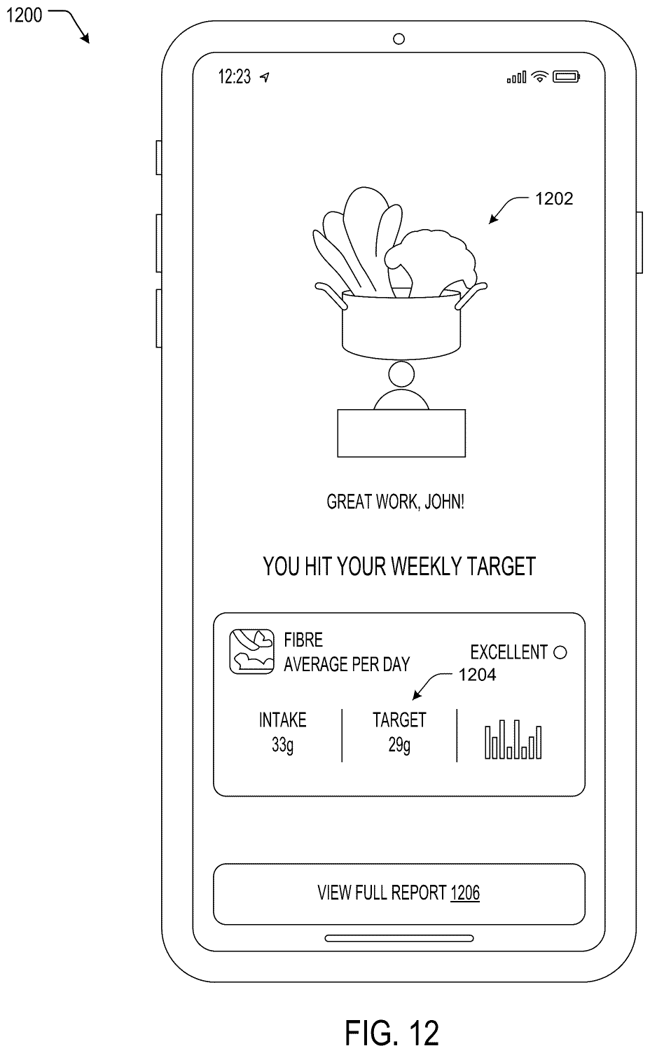

FIG. 12 is an example pictorial diagram illustrating an example user interface 1200 of a notification 1202 associated with a personalized target for a given period of time according to some implementations. For example, the current notification 1202 and user interface 1200 may be responsive to a participant achieving or completing the current target 1204. The current user interface 1200 associated with the notification 1202 may include a user selectable option 1206 to receive more information or a full report of the consumption data associated with the target 1204.

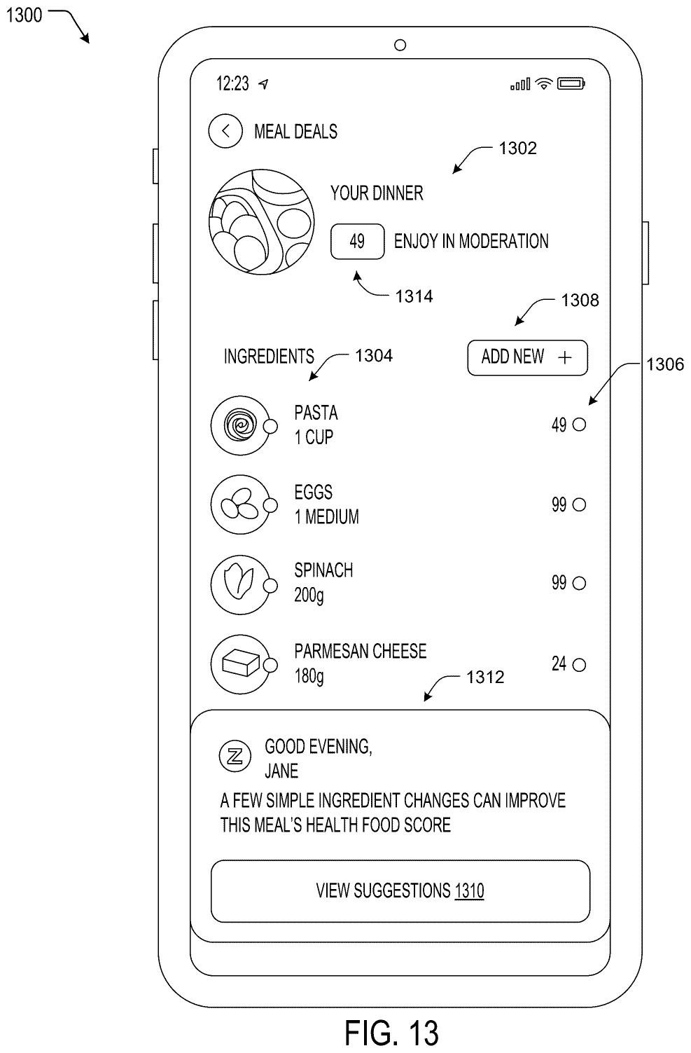

FIG. 13 is an example pictorial diagram illustrating an example user interface 1300 associated with health food score for a meal plan option 1302 (in the current example dinner) including each ingredient food score personalized for the participant according to some implementations. In the current example, the meal plan option 1302 includes a list of ingredients 1304 with a corresponding food score, generally indicated by 1306, for each of the ingredients 1304. As discussed herein, the food score 1306 for each ingredient 1304 is personalized for the individual participant.

The user interface 1300 may also include a selectable option 1308 to add an additional ingredient to the list 1306. Likewise, in some cases, the participant may be able to select an ingredient and remove the ingredient from the list 1306. The user interface 1300 may also include a notification or recommendation 1312 to allow the participant to boost or improve their health food score 1314 of the overall meal, such as by selecting the option 1310, to show the meal plan edits recommended by the system.

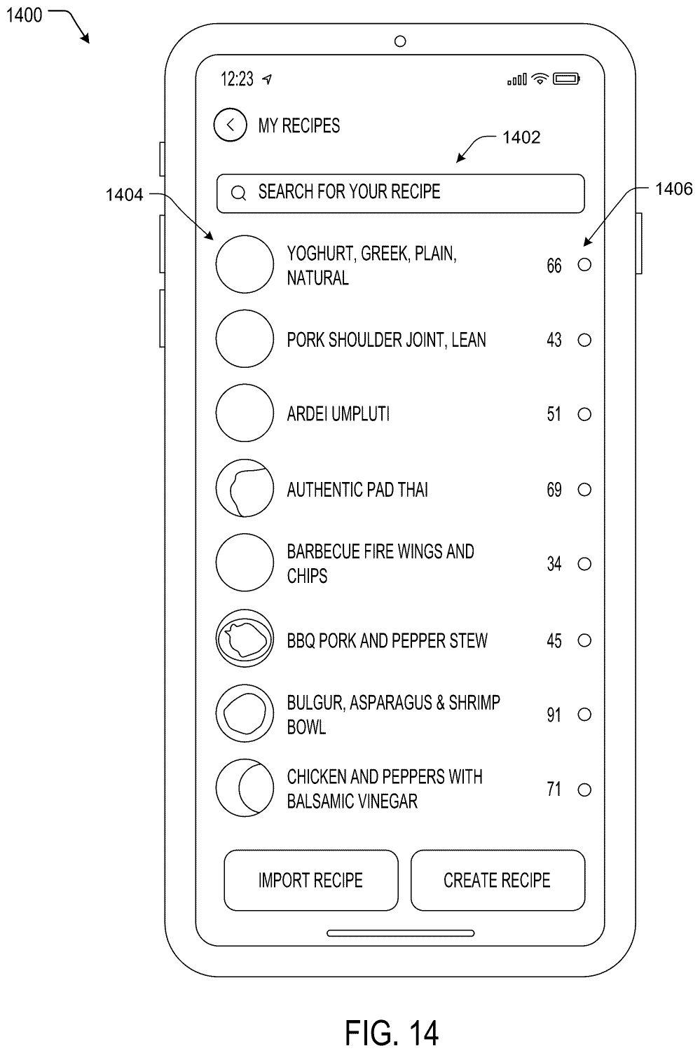

FIG. 14 is an example pictorial diagram illustrating an example user interface 1400 associated with generating meal plan options or recipes according to some implementations. In the current example, the user interface 1400 may include a search option 1402 to allow a participant to enter a free form text based search for meal plan options. The meal plan options may be selected or generated based at least in part on the user input into the search option 1402 as well as the food scores, health food scores, and participant data associated with the participant. In some cases, the meal plan options may be selected or generated based on the meal (e.g., breakfast, lunch, dinner, snack, or the like), preferences of the participant, taste profiles associated with the participant, season of the year, cooking preferences of the participant, other meal plan options for proximate meals (e.g., selection of different proteins for lunch and dinner on the same day), planed shopping list or receipt data, available food items in a food storage area, and the like.

Once generated, the user interface 1400 may provide a list of meal plan options 1404 and a corresponding health food score, generally indicated by 1406, for each option of the list 1404. In some cases, each meal plan option is selectable such that the participant may view in more detail, boost the meal plan option, edit the meal plan option, and/or the like.

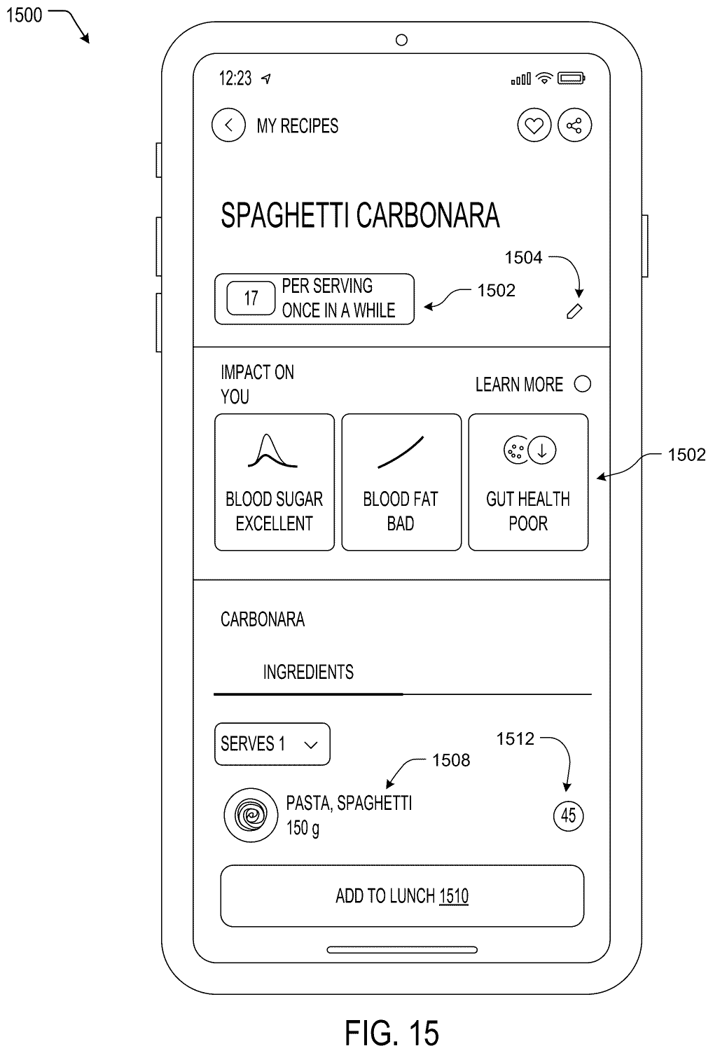

FIG. 15 is another example pictorial diagram illustrating an example user interface 1500 associated with generating meal plan options (such as Spaghetti Carbonara) according to some implementations. In the current user interface 1500, the meal plan option is shown with a current health food score 1502 and a selectable option 1504 to edit the meal plan option. The user interface 1500 may also include indicators 1506 of a health impact of the meal personalized for the participant. The participant may also be able to view the ingredients 1508, a corresponding food score 1512, and to select the meal plan option for a specific meal, via selectable option 1510.

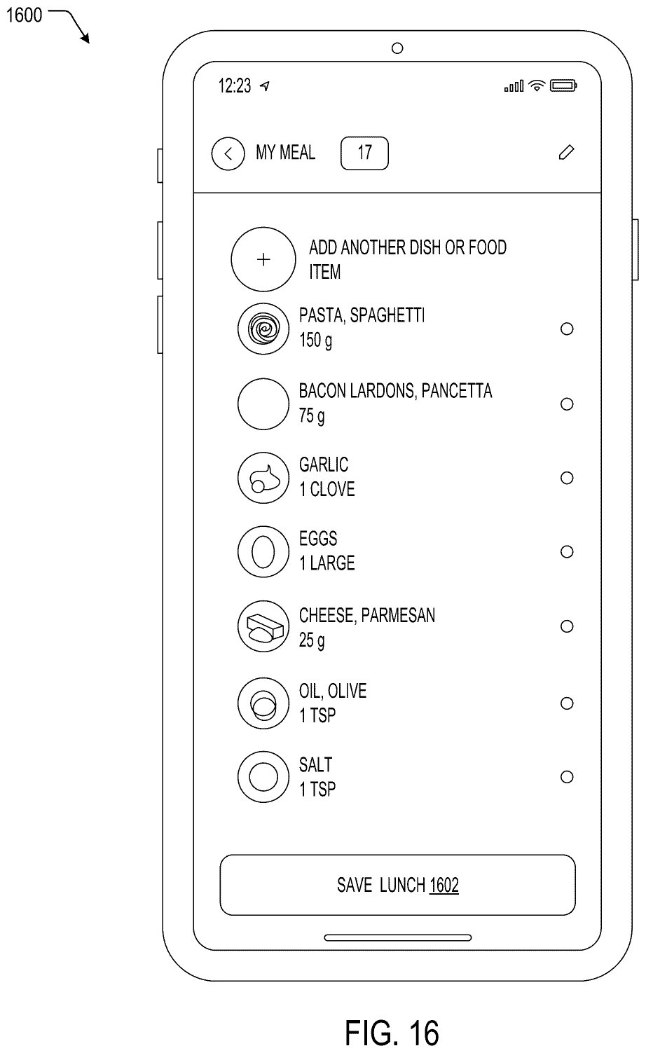

FIG. 16 is another example pictorial diagram illustrating an example user interface 1600 associated with generating meal plan options according to some implementations. In the current example, the participant may view a current meal plan option and save the option to a specific meal, such as lunch via the selectable option 1602.

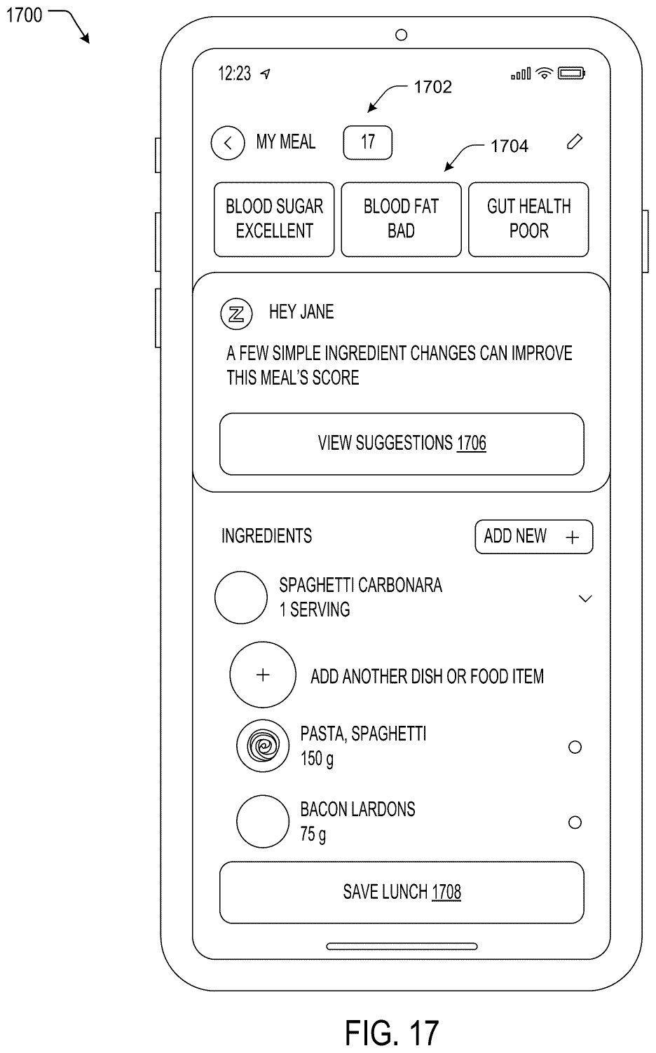

FIG. 17 is an example pictorial diagram illustrating an example user interface 1700 associated with boosting a meal plan option according to some implementations. In the current example, the user interface 1700 may include the meal and the corresponding health food score 1702 as well as the meal plan options health benefits 1704. The user interface 1700 may also include a selectable option 1706 to boost the meal plan option in a personal way for the participant, as discussed herein. In the current example, the participant may also save the option to a specific meal, such as lunch via the selectable option 1708.

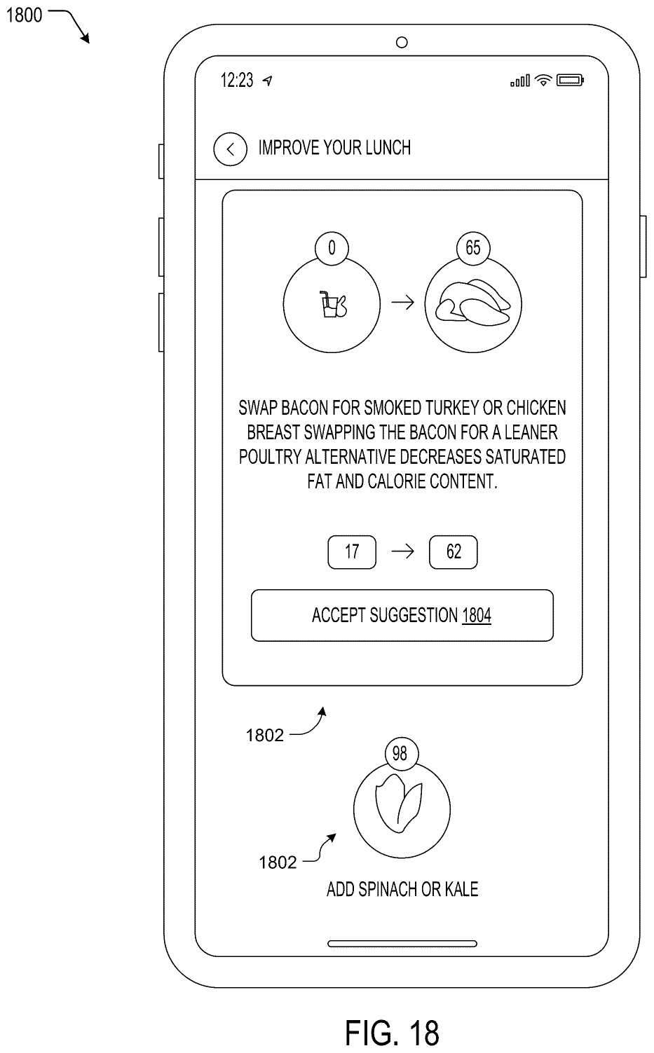

FIG. 18 is another example pictorial diagram illustrating an example user interface 1800 associated with boosting a meal plan options according to some implementations. For example, the participant may have selected the option 1706 in the user interface 1700 discussed above and been transitioned to the user interface 1800. The current user interface 1800, a recommended boost is shown in a recommendation display area 1802. For instance, as illustrated, the system is recommending swapping bacon for turkey or chicken breast. The recommendation display area 1802 may also present the current health food score of the meal plan options and the updated health food score after accepting the recommendation, here the swap of bacon for turkey or chicken. In the current example, the participant may accept the swap via the accept suggestion selectable option 1804.

In the current user interface 1800, the system may also present an option for a secondary or any number of additional boosts. As illustrated, the user interface 1800 may include a selectable option 1806 to add spinach or kale to the meal plan option via a second boost to reach a health food score of 98.

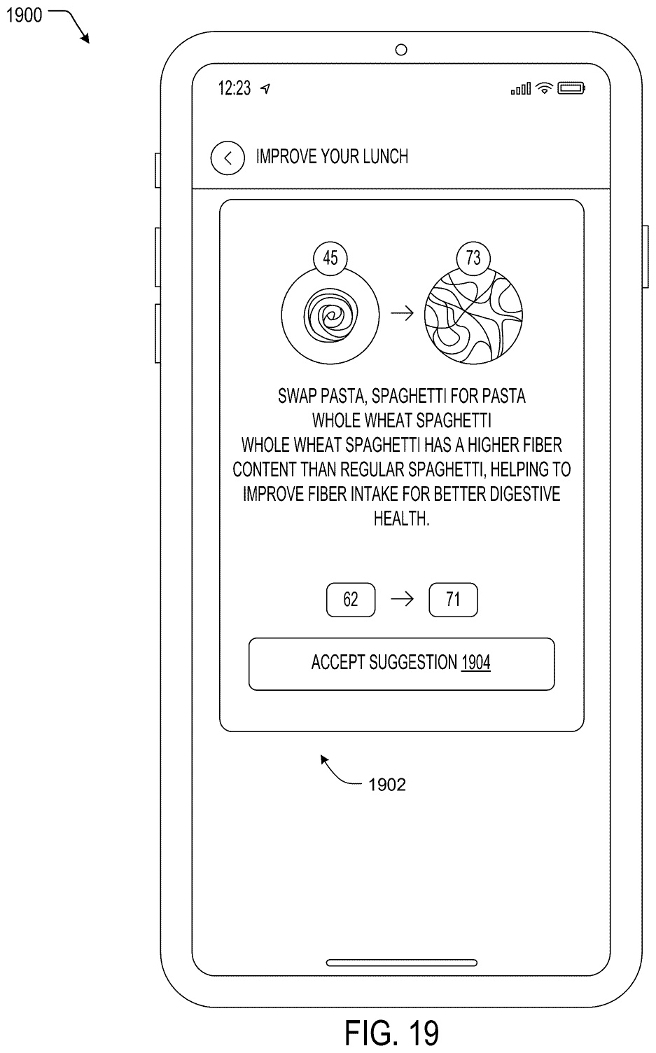

FIG. 19 is another example pictorial diagram illustrating an example user interface 1900 associated with boosting a meal plan options according to some implementations. For example, the participant may have selected the option 1706 in the user interface 1700 discussed above and been transitioned to the user interface 1900. The current user interface 1900, a recommended boost is shown in a recommendation display area 1902. For instance, as illustrated, the system is recommending swapping spaghetti pasta to whole wheat spaghetti. The recommendation display area 1902 may also present the current health food score of the meal plan options and the updated health food score after accepting the recommendation, here the swap of spaghetti pasta to whole wheat spaghetti. In the current example, the participant may accept the swap via the accept suggestion selectable option 1904.

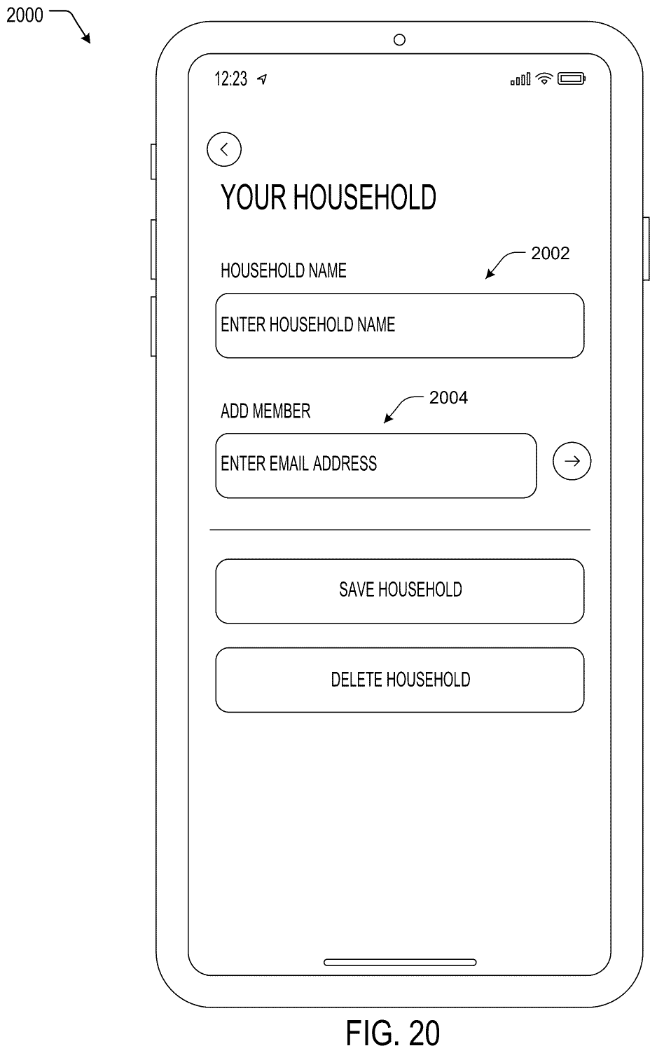

FIG. 20 is an example pictorial diagram illustrating an example user interface 2000 associated with managing a group according to some implementations. As discussed above, a group may include two or more participants that share meals. In the current example, a participant may create a group via option 2002 or add members via option 2004.

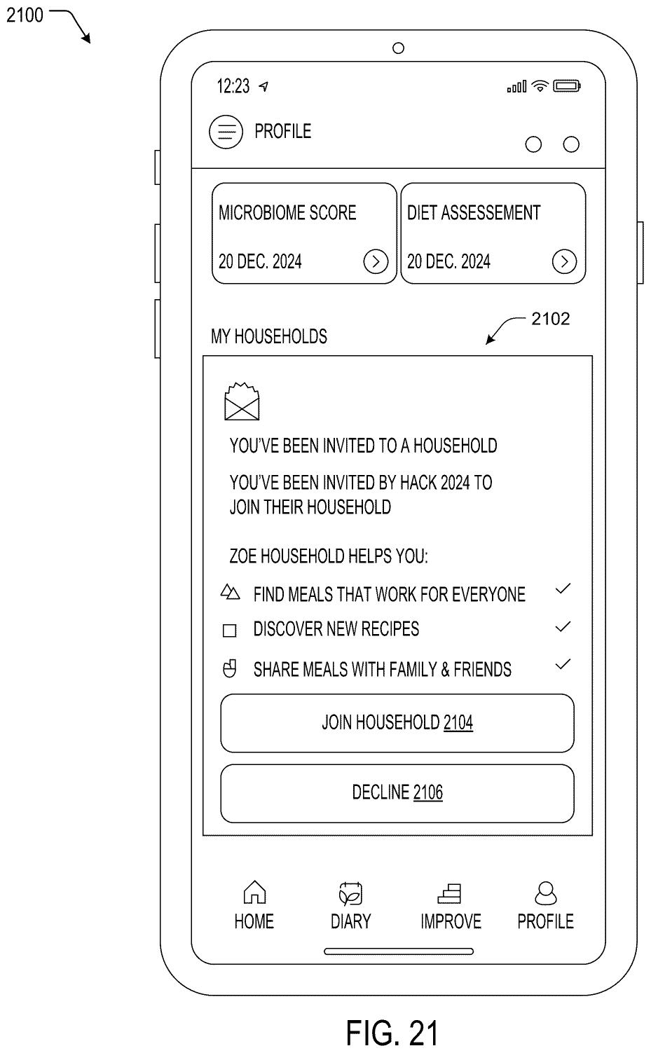

FIG. 21 is an example pictorial diagram illustrating an example user interface 2100 associated with joining a group according to some implementations. For example, if a participant was invited to be a member of a group via user interface 2000, the invited participant may receive a notification 2102 as shown. Via the notification 2102, the invited participant may join via option 2104 or decline via option 2106.

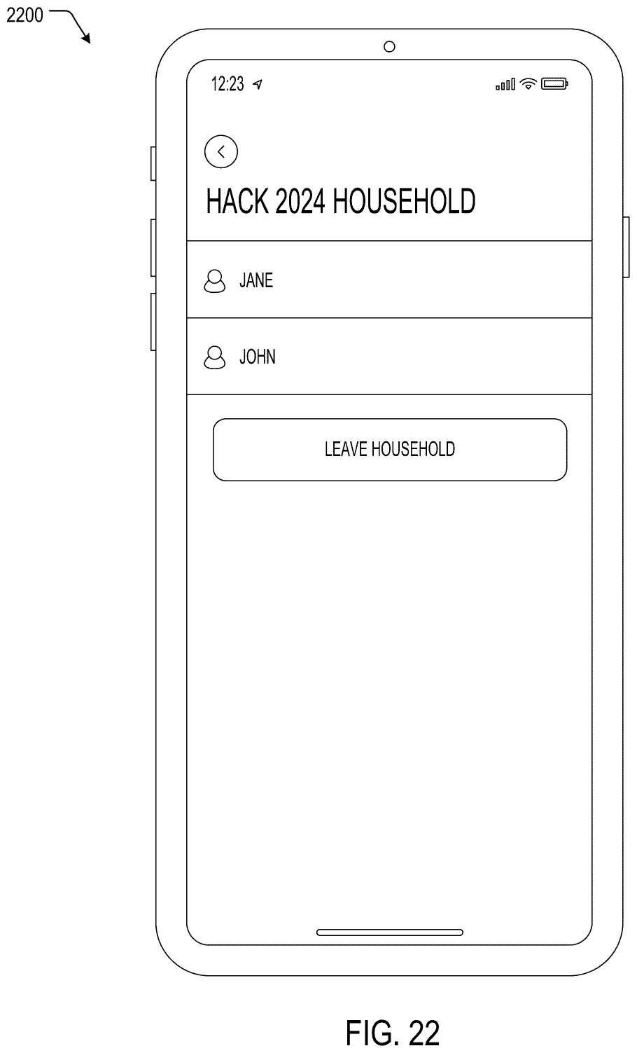

FIG. 22 is another example pictorial diagram illustrating an example user interface 2200 associated with managing a group according to some implementations. In the current example, once a participant has joined a group, the user interface 2200 may allow the participant to view each member of the group, as shown.

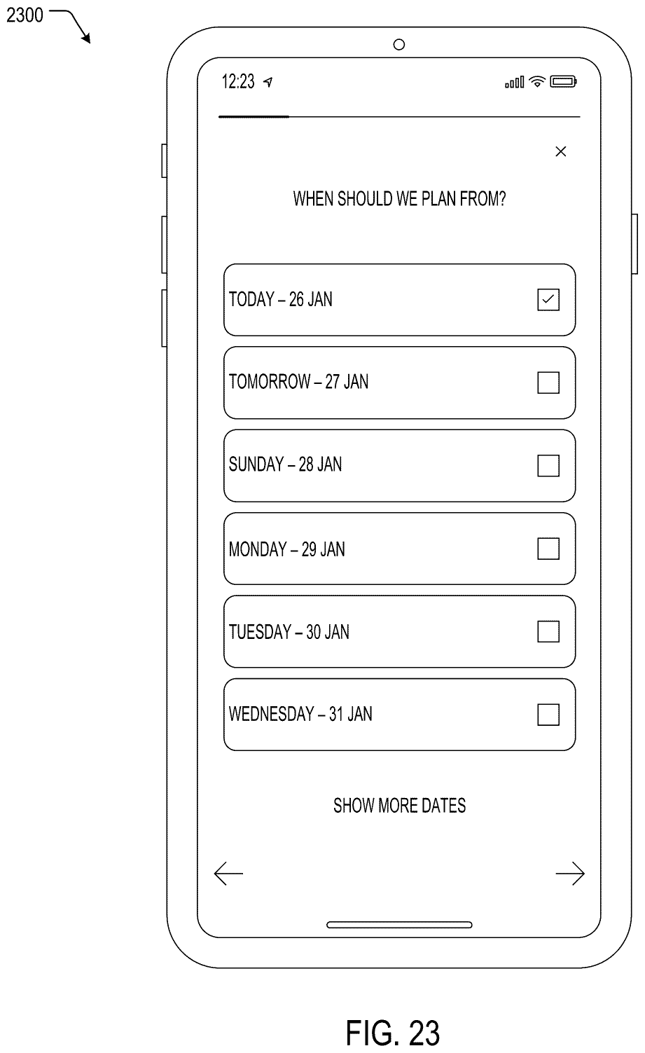

FIG. 23 is an example pictorial diagram illustrating an example user interface 2300 associated with generating a meal plan options for a given period of time according to some implementations. For example, in the current example, a participant may select a date and time at which to start the given period of time for a meal plan generator session.

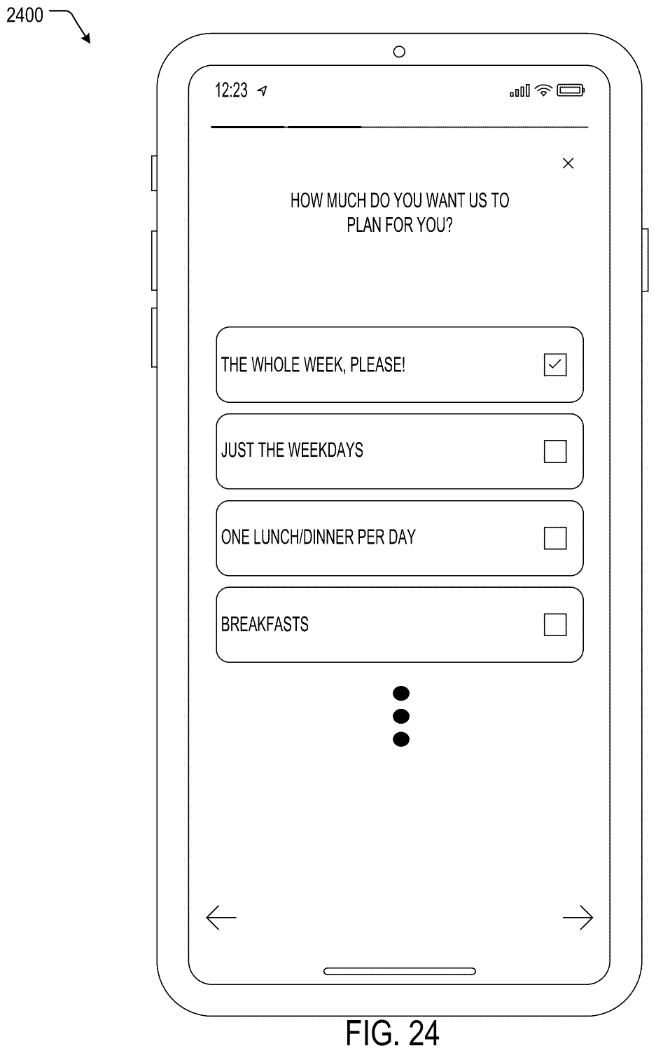

FIG. 24 is another example pictorial diagram illustrating an example user interface 2400 associated with generating a meal plan options for a given period of time according to some implementations. For example, using the interface 2400, the participant may select which meals the system should include in the meal plan option generator session. For instance, the participant may select every meal, the weekday meals, lunch and dinner but not breakfast, breakfast only, and/or other options (not shown).

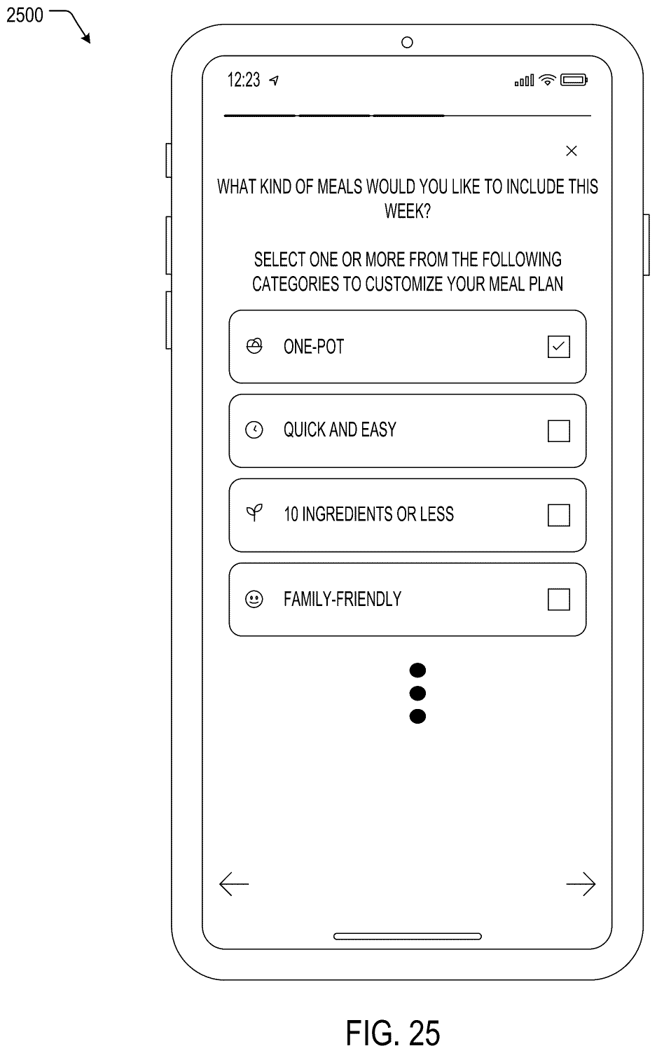

FIG. 25 is another example pictorial diagram illustrating an example user interface 2500 associated with generating a meal plan options for a given period of time according to some implementations. In the current example, the participant may also provide additional input or direction into the meal plan option generation process. For example, the participant may select to limit meals to a single pot, easy to prepare (or other difficulty level), a number of ingredients, a range of tastes (such as family friendly to remove overly spicy recipes for kids), and/or other options (not shown, for example preferred cuisines or ingredients).

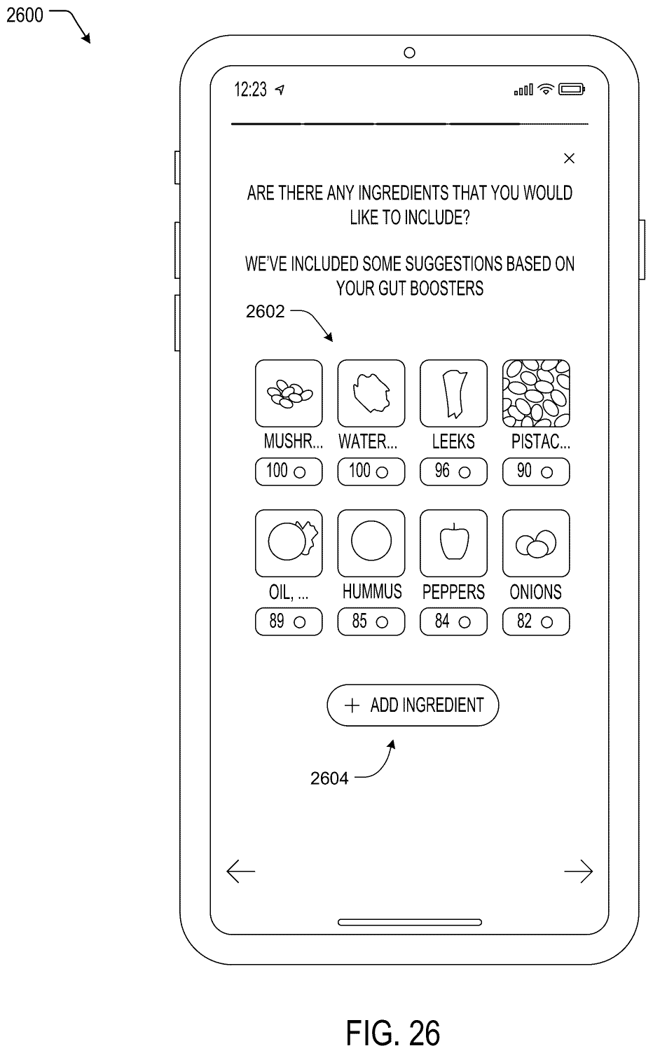

FIG. 26 is another example pictorial diagram illustrating an example user interface 2600 associated with generating a meal plan options for a given period of time according to some implementations. In the current example, the participant may select or otherwise indicate specific ingredients to include in the meal plan generation process. For example, the user may select various ingredients that are recommended by the system, generally indicated by 2602, or via the add ingredient of their choice via option 2604.

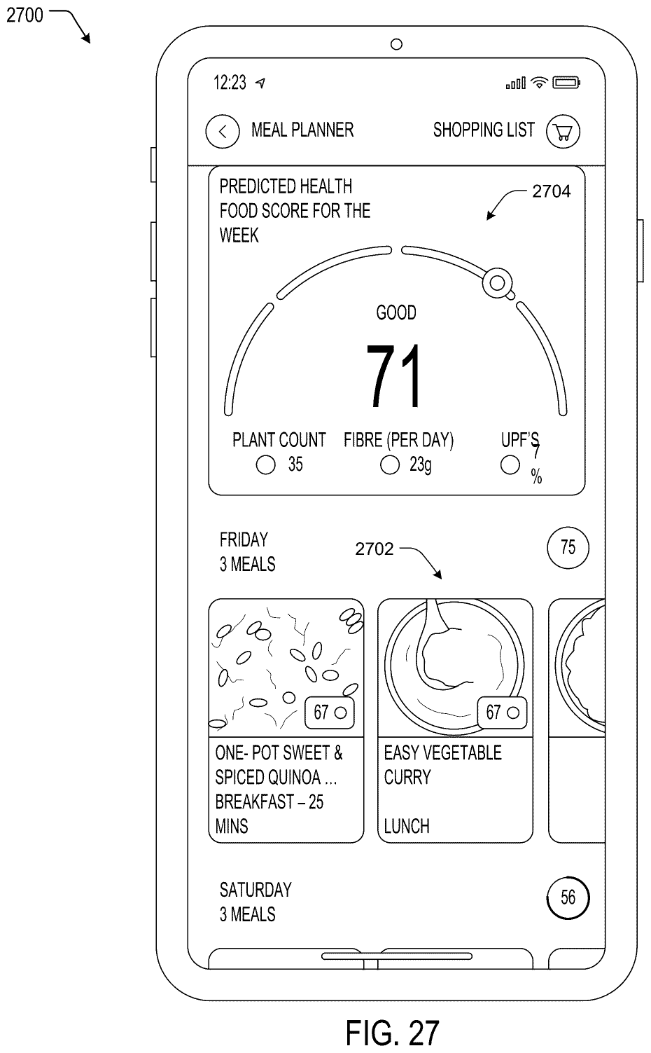

FIG. 27 is another example pictorial diagram illustrating an example user interface 2700 associated with generating a meal plan options for a given period of time according to some implementations. In the current example, the system may present to the participant the meal plan options 2702 for the given period of time (e.g., the week) on a daily basis. The user interface 2700 may also include the predicted health food score for the time period via area 2704.

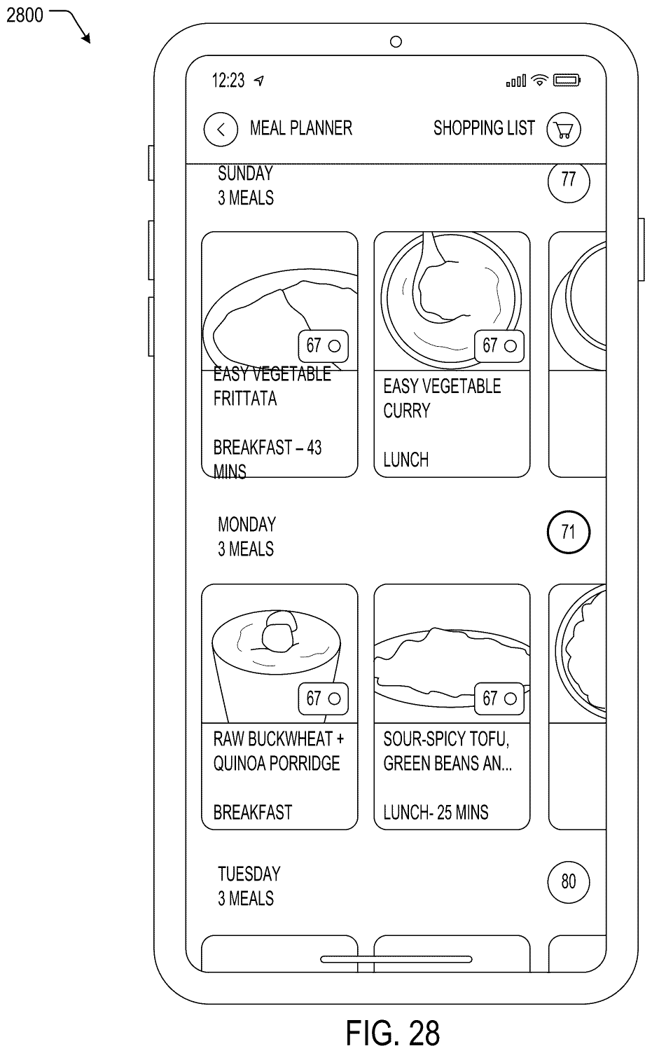

FIG. 28 is another example pictorial diagram illustrating an example user interface 2800 associated with generating a meal plan options for a given period of time according to some implementations. In the current example, the user may have vertically scrolled the display of FIG. 27 to see additional meal plan options for additional days of the period of time. In this manner, the participant may review, select, and modify any of the meal plan options for the given period of time.

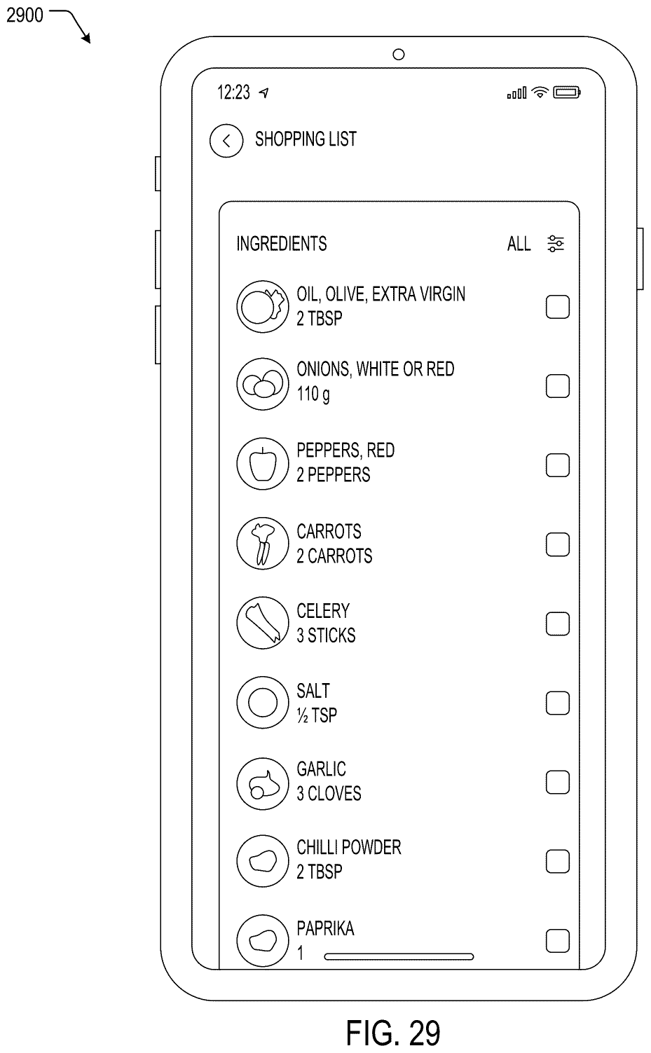

FIG. 29 is an example pictorial diagram illustrating an example user interface 2900 associated with generating a shopping list for a given period of time according to some implementations. For example, the system may generate the shopping list based on the meal plan options generated such as via the interfaces of FIGS. 23-28 above.

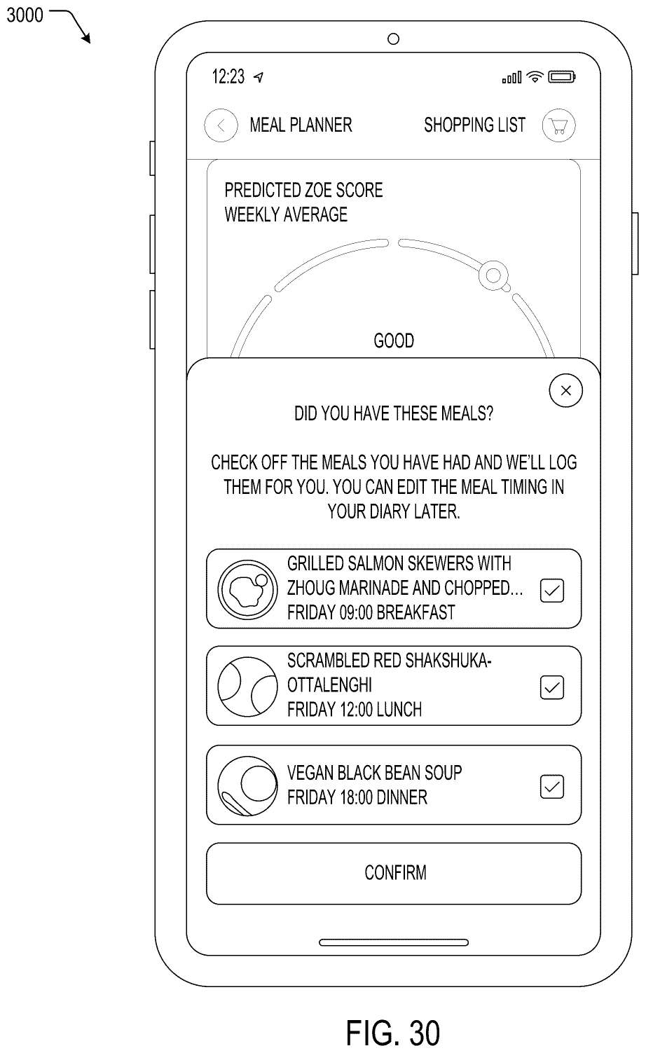

FIG. 30 is an example pictorial diagram illustrating an example user interface 3000 associated with logging consumption data for a given period of time according to some implementations. For example, the system may log consumption data based at least in part on the meal plan options associated with a given period of time and generated by the user interfaces of FIGS. 230-28 above and/or the shopping list of FIG. 29. In the current example, the system may also present the participant the option to confirm consumption of each meal prior to logging the associated consumption data.

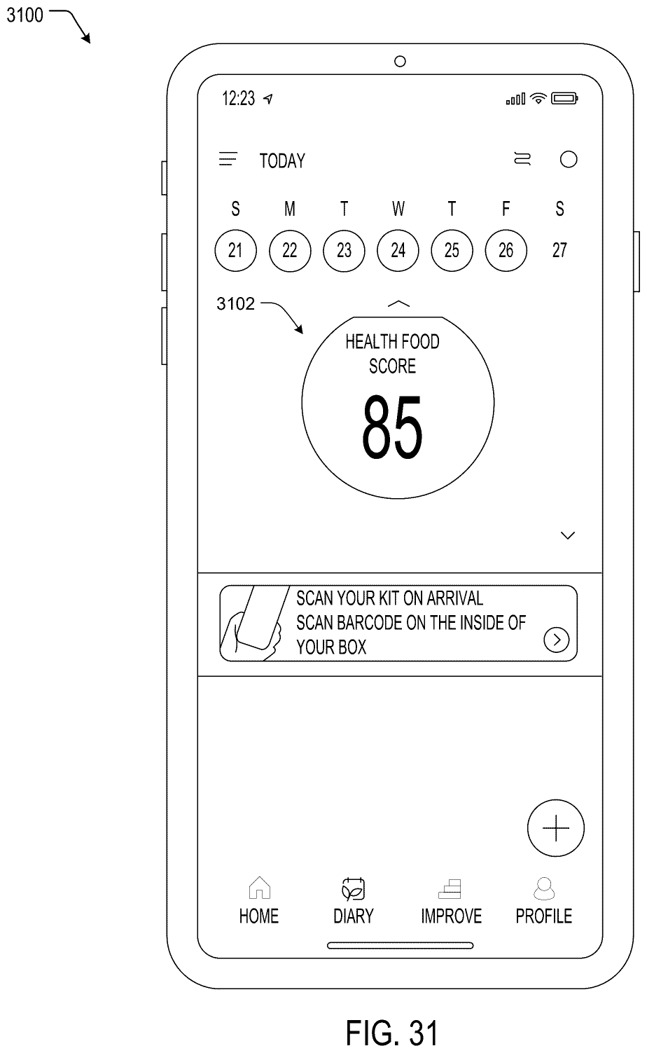

FIG. 31 is an example pictorial diagram illustrating an example user interface 3100 associated with a personalized health food score 3102 for a given period of time according to some implementations.

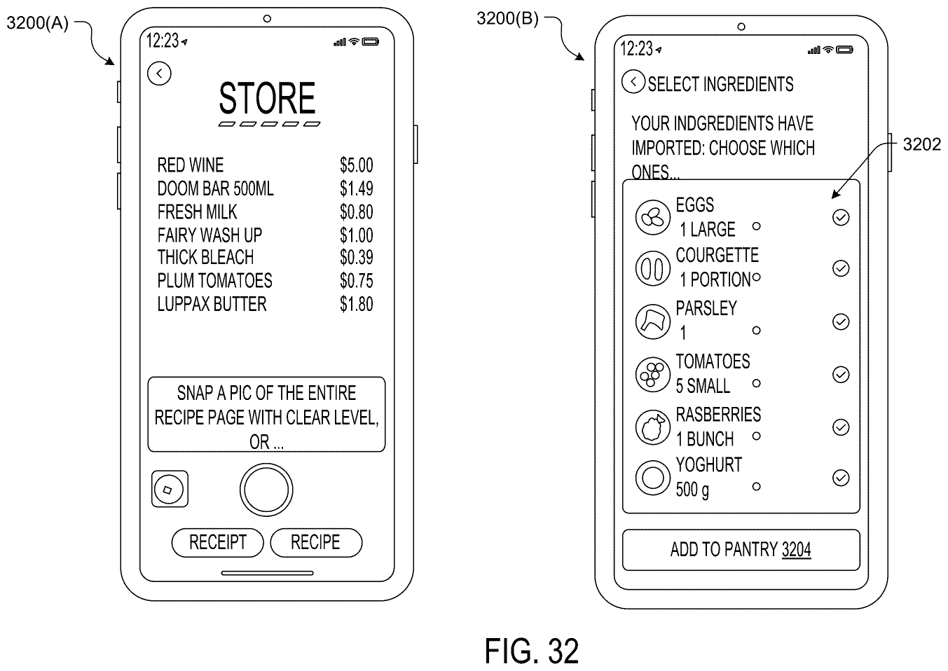

FIG. 32 is an example series of pictorial diagrams illustrating an example user interface 3200(A) and 3200(B) associated with scanning a receipt and logging consumption data for a given period of time according to some implementations. In the user interface 3200(A) a user has captured via an image a copy of the receipt for a shopping event. At user interface 3200(B) the system may present a list of detected food items from the receipt of 3200(A). The participant may then select and add the food items to the pantry using options 3202 and 3204 interface 3200(B).

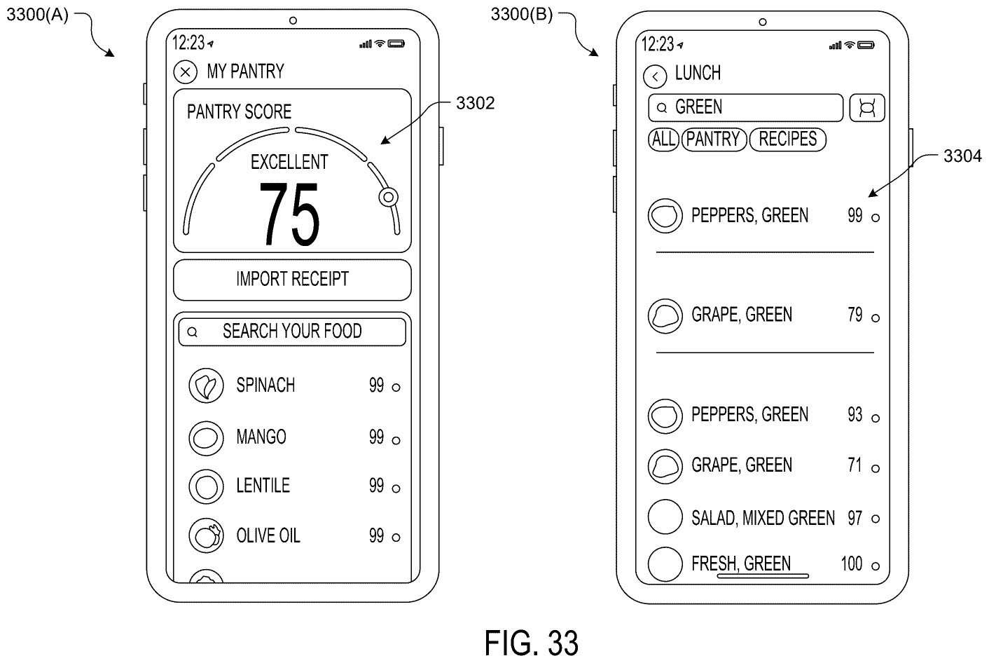

FIG. 33 is an example series of pictorial diagrams illustrating an example user interface 3300(A) and 3300(B) associated with scanning a receipt and logging consumption data for a given period of time according to some implementations. The user interface 3300(A), the system may present a health food score or pantry score 3302 for the items added to the pantry at 3200(B). The interface 3300(B) may allow the participant to search the pantry items, such as by searching for the word green to receive a displayed list of green items 3304.

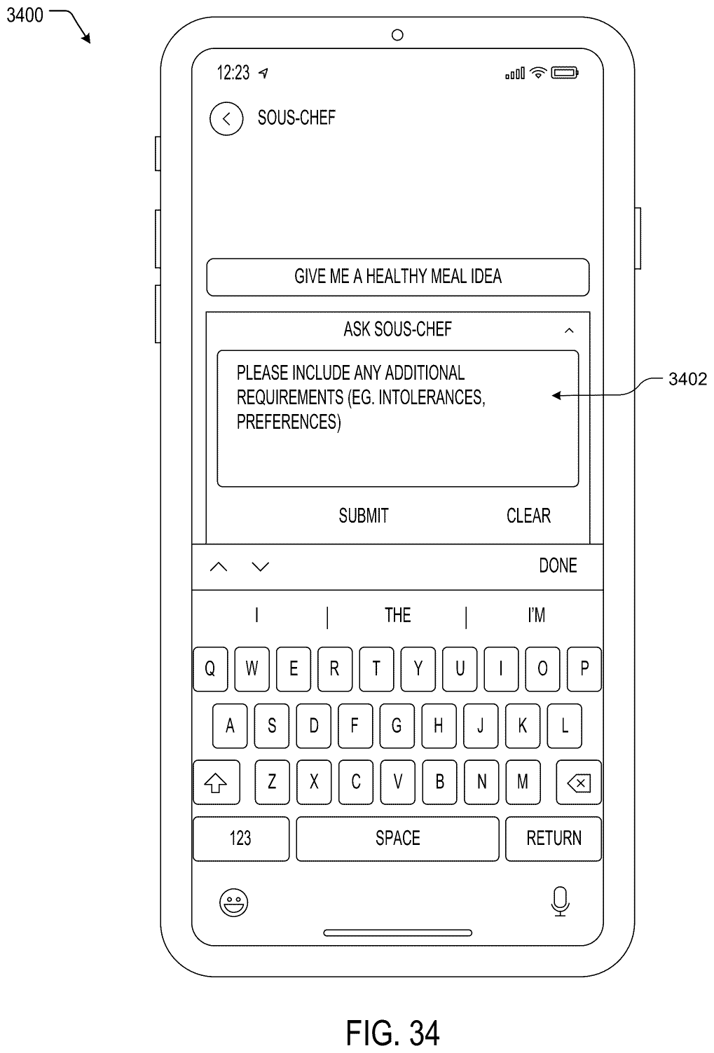

FIG. 34 is an example pictorial diagram illustrating an example user interface 3400 associated with generating a contextual based meal plan option for a given meal according to some implementations. In the current example, a participant may enter a free form text-based input into text input area 3402 in order for the system to generate a meal plan option based on the text-based input.

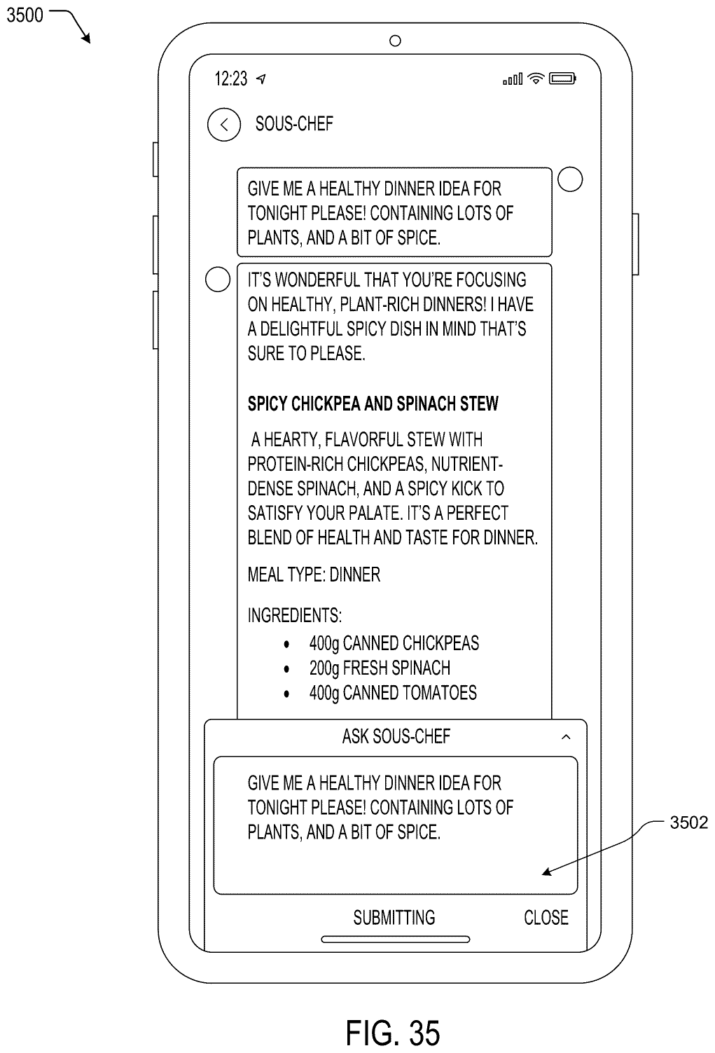

FIG. 35 is another example pictorial diagram illustrating an example user interface 3500 associated with generating a contextual based meal plan option for a given meal according to some implementations. In the current example, the system may present to the participant a meal plan option including the recipe based on the text-based input of 3402. In the current example, the text-based input may be displayed in the area 3502 which may also be utilized to enter additional information for updating the meal plan option.

Although the discussion above sets forth example implementations of the described techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

### EXAMPLE CLAUSES

A. A method comprising: generating, based at least in part on first participant data associated with a first participant, a meal plan option for the first participant, the meal plan option designed to be healthy for the first participant; determining, based at least in part on the first food item and first participant data associated with the first participant, a first health food score of the meal plan option; and presenting the meal plan option and the first health food score on a display of the user device.

B. The method of claim A, further comprising: receiving sensor data from a user device associated with the first participant; determining, based at least in part on the sensor data, an identity of one or more food items; and generating the meal plan option is based at least in part on data associated with available food items in a food storage area associated with the first participant, wherein the meal plan option includes at least one of the available food items from the food storage area or the one or more identified food items.

C. The method of claim A, wherein the first participant data includes one or more of microbiome data associated with a gut of the first participant, allergy data associated with the first participant, and health data associated with the first participant.

D. The method of claim A, wherein generating the meal plan option is based at least in part on one or more of: a dietary preference of the first participant; or responsive to a user input on a user interface of an application hosted on a user device.

E. The method of claim A, wherein presenting the meal plan option and the first health food score on the display of the user device further comprises presenting a recipe or cooking and preparation instruction on the display.

F. The method of claim A, further comprising at least one of: responsive to determining a period of time associated with the meal plan option has elapsed, logging consumption data associated with the meal plan on behalf of the first participant; or responsive to receiving a user input associated with the meal plan option, logging consumption data associated with the meal plan on behalf of the first participant.

G. The method of claim A, wherein the first participant is associated with a group of participants having a second participant and the method further comprises: determining, based at least in part on the first food item and second participant data associated with the second participant, a second health food score of the meal plan option; and responsive to determining that the first health food score meet or exceed a first threshold and the second health food score meet or exceed a second threshold, presenting the second health food score with the meal plan option on the display of the user device.

H. The method of claim A, wherein the meal plan option is a first meal plan option associated with a meal of a given period of time and the method further comprises: determining, based at least in part on the first meal plan option, remaining food items; generating, based at least in part on the remaining food items, at least one second meal plan option for the first participant, each meal plan option of the at least one second meal plan option associated with a given period of time and a particular meal of the given period of time; determining, based at least in part on the first participant data associated with the first participant, at least one second health food score for each of the at least one second meal plan option; determining, based at least in part on the first health food score and the at least one second health food score, a total health food score for the given period of time; and presenting the at least one second meal plan option, the at least one second health food score, and the total health food score on the display of the user device together with the first health food score and the first meal plan option.

I. A system comprising: one or more processors; and one or more non-transitory computer readable media storing instructions executable by the one or more processors, wherein the instruction, when executed, cause the one or more processors to perform operations comprising: generating, based at least in part on first participant data associated with a first participant and a given period of time, one or more meal plan options for each meal associated with the given period of time, the meal plan options designed to be healthy for the first participant; determining, for each meal plan option associated with the given period of time and based at least in part on food items associated with each meal plan option and first participant data associated with the first participant, a first health food score for each meal plan option for the first participant; and causing the meal plan options and the first health food score for each meal plan option to be presented on a display of a user device associated with the first participant.

J. The system of claim 1, wherein the operations further comprise: determining, based at least in part on the first health food score for each meal plan option, a total health food score for the given period of time; and causing the total health food score to be presented on a display of a user device associated with the first participant.

K. The system of claim 1, wherein the operations further comprise at least one of: generating a shopping list of food items to be purchased for the meal plan options; or ordering at least one of the food items from a third-party grocery delivery service.

L. The system of claim 1, wherein the operations further comprise: determining, for each meal plan option a second health food score for each meal plan for a second participant; and filtering the meal plan options based at least in part on a first threshold health food score associated with the first participant and a second threshold health food score associated with the second participant.

M The system of claim 1, wherein: the first participant data includes one or more of microbiome data associated with a gut of the first participant, allergy data associated with the first participant, and health data associated with the first participant; and the microbiome data includes at least one gut booster food or at least one gut suppressor food.

N. One or more non-transitory computer readable media storing instructions executable by one or more processors, wherein the instruction, when executed, cause the one or more processors to perform operations comprising: receiving shopping data associated with a first participant; determining food items purchased by the first participant from the shopping data; determining, for each food item purchased, a food score representing a health of the food item for the participant based at least in part on participant data associated with the participant; and causing the food scores to be presented on a display of the user device associated with the participant.

O. The one or more non-transitory computer readable media of claim N, wherein the shopping data is at least one of: image data from a user device associated with the first participant; and the image data is segmented and classified to determine food items purchased by the first participant; or electronic data associated with a third-party grocery delivery service.

P. The one or more non-transitory computer readable media of claim N, wherein the operations further comprise: logging at least one of the food items as consumption data associated with a period of time on behalf of the participant.

Q. The one or more non-transitory computer readable media of claim N, wherein: a health food score associated with the shopping data is determined based at least in part on the food scores.

R. The one or more non-transitory computer readable media of claim of claim Q, wherein: the health food score is a first health food score and is associated with a first period of time; and the operations further comprise: determining change data between the first health food score and a second health food score associated with a second period of time prior to the first period of time; and causing the change data to be presented on the display of the user device associated with the participant.

S. The one or more non-transitory computer readable media of claim N, wherein the operations further comprise: causing advice for future food shopping to be presented on a display of the user device associated with the participant, the advice based at least in part of the food scores, wherein the advice may include at least one of: food items to continue or increase purchasing; new food items to start purchasing; food items to decrease or stop purchasing; and food items to decrease or stop purchasing and replace with alternative food items.

T. The one or more non-transitory computer readable media of claim N, wherein the operations further comprise: determining, based at least in part on the food scores and additional food scores associated with known available food items in a food storage area associated with the participant, a pantry score; and causing the pantry score to be presented on the display of the user device associated with the participant.

U. A method comprising: presenting an initial meal plan option and an initial health food score associated with the meal plan option and a participant on a display of a user device; generating, based at least in part on the initial meal plan option, an associated second meal plan option, the second meal plan option having a second health food score for the participant greater than the initial health food score associated with the initial meal plan option; and presenting the second meal plan option on the display of the user device.

V. The method of claim U, further comprising: receiving sensor data from a user device associated with a first participant; determining, based at least in part on the sensor data, an identity of one or more food items; and generating, based at least in part on the identity of the one or more food items, the second meal plan option for the participant.

W. The method of claim U, further comprising: generating the second meal plan option for the participant based at least in part on data associated with available food items in a food storage area associated with the participant.

X. The method of claim U, wherein the user interface and the display are a touch enabled display.

Y. The method of claim U, wherein generating the second meal plan option further comprises; selecting a first food item to swap with a second food item associated with the initial meal plan option, and wherein: the first food item having a first food score greater than a second food score associated with the second item; the first food score and the second food score are personalized for the participant; and the first food item is a food item suitable for use in a recipe associated with the initial meal plan option.

Z. The method of claim U, further comprising: determining change data between the second health food score and the initial health food score; and causing the change data to be presented on the display of the user device associated with the participant together with the second health food score and the second meal plan option.

AA. The method of claim U, further comprising: generating, based at least in part on the second meal plan option, an associated third meal plan option, the third meal plan option having a third health food score for the participant greater than the second health food score associated with the second meal plan option; and presenting the third meal plan option on the display of the user device.

AB. The method of claim U, wherein generating the second meal plan option is based at least in part on one or more of the following: one or more of microbiome data associated with a gut of the participant, allergy data associated with the participant and health data associated with the participant; or at least in part on a dietary preference of the participant.

AC. The method of claim U, further comprising: receiving, from the user interface of the user device, an approval of the second meal plan option; and adding the second meal plan option to a meal plan for a given period of time.

AD. A method comprising: determining a period of time; determining, based at least in part on participant data associated with a participant and a health goal associated with the participant, one or more targets for the period of time; presenting the one or more targets for the period of time on a display of a user device associated with the participant; receiving consumption data associated with food items consumed during the period of time; generating, based at least in part on the consumption data, progress data associated with the one or more targets; and sending, via the user device, a notification associated with the progress data to the participant.

AE. The method of claim AD, further comprising: receiving a user selection of one or more selected targets from a presented list of possible targets for the period of time.

AF. The method of claim AD, further comprising presenting on the user device one or more of: relevant educational material associated with the one or more targets; and relevant recommended actions associated with the one or more targets.

AG. The method of claim AD, wherein determining the one or more targets for the period of time is based at least in part on one or more of: prior progress data associated with a prior target associated with a second period of time prior to the period of time; a prior health food score associated with a second period of time prior to the period of time; a biology of the participant determined from sensor data associated with the participant or from one or more tests performed on the participant; user inputs responsive to one or more questionnaires presented to the participant via the user device; or a current diet of the participant determined based at least part on consumption data associated with the participant over prior period of time.

AH. The method of claim AD, further comprising receiving the health goal as a user input from a user interface of a user device associated with the participant.

AI. The method of claim AD, wherein sending the notification associated with the progress data to the participant is responsive to determining that the progress data meets or exceeds one or more thresholds.

AJ. The method of claim AD, wherein the target is a primary target and the method further comprises: determining, based at least in part on the period of time, participant data associated with a participant, a health goal associated with the participant, and the primary target, a first intermediate target for a first subset of the period of time and a second intermediate target for a second subset of the period of time, the second subset different than the first subset; receiving consumption data associated with food items consumed during the period of time; determining, based at least in part on the consumption data, progress data associated with the first intermediate target or the second intermediate target; and sending, via the user device, a notification to the participant in response to the progress data indicating a completion of the first intermediate target or the second intermediate target.

AK. The method of claim AD, further comprising: responsive to determining that the progress data meets or exceeds one or more progress thresholds and the period of time is less than or equal to a time threshold, determining an update to the target, and sending, via the user device, a second notification associated with the update to the target.

AL. The method of claim AD, wherein the participant data includes one or more of microbiome data associated with a gut of the participant, allergy data associated with the participant and health data associated with the participant.

AM. A system comprising: one or more processors; and one or more non-transitory computer readable media storing instructions executable by the one or more processors, wherein the instruction, when executed, cause the one or more processors to perform operations comprising: determining, based at least in part on participant data associated with a participant and a health goal associated with the participant, one or more targets for a period of time; receiving consumption data associated with food items consumed during the period of time; generating, based at least in part on the consumption data, progress data associated with the one or more targets; and sending a notification associated with the progress data to the participant.

AN. The system of claim AM, further comprising: determining, based at least in part on the progress data, a completion metric associated with the one or more targets; and wherein the notification includes the completion metric.

While the example clauses described above are described with respect to one particular implementation, it should be understood that, in the context of this document, the content of the example clauses can also be implemented via a method, device, system, a computer-readable medium, and/or another implementation. Additionally, any of examples A-AN may be implemented alone or in combination with any other one or more of the examples A-AN.

### CONCLUSION

While one or more examples of the techniques described herein have been described, various alterations, additions, permutations and equivalents thereof are included within the scope of the techniques described herein. As can be understood, the components discussed herein are described as divided for illustrative purposes. However, the operations performed by the various components can be combined or performed in any other component. It should also be understood that components or steps discussed with respect to one example or implementation may be used in conjunction with components or steps of other examples.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

What is claimed is:

1. A method comprising:
presenting an initial meal plan option and an initial health food score associated with the meal plan option and a participant on a display of a user device;
generating, based at least in part on the initial meal plan option, an associated second meal plan option, the second meal plan option having a second health food score for the participant greater than the initial health food score associated with the initial meal plan option; and
presenting the second meal plan option on the display of the user device.

2. The method of claim 1, further comprising:
receiving sensor data from a user device associated with a first participant;
determining, based at least in part on the sensor data, an identity of one or more food items; and
generating, based at least in part on the identity of the one or more food items, the second meal plan option for the participant.

3. The method of claim 1, further comprising:
generating the second meal plan option for the participant based at least in part on data associated with available food items in a food storage area associated with the participant.

4. The method of claim 1, wherein the user interface and the display are a touch enabled display.

5. The method of claim 1, wherein generating the second meal plan option further comprises;
selecting a first food item to swap with a second food item associated with the initial meal plan option, and wherein:
the first food item having a first food score greater than a second food score associated with the second item;
the first food score and the second food score are personalized for the participant; and
the first food item is a food item suitable for use in a recipe associated with the initial meal plan option.

6. The method of claim 1, further comprising:
determining change data between the second health food score and the initial health food score; and
causing the change data to be presented on the display of the user device associated with the participant together with the second health food score and the second meal plan option.

7. The method of claim 1, further comprising:
generating, based at least in part on the second meal plan option, an associated third meal plan option, the third meal plan option having a third health food score for the participant greater than the second health food score associated with the second meal plan option; and
presenting the third meal plan option on the display of the user device.

8. The method of claim 1, wherein generating the second meal plan option is based at least in part on one or more of the following:
one or more of microbiome data associated with a gut of the participant, allergy data associated with the participant and health data associated with the participant; or
at least in part on a dietary preference of the participant.

9. The method of claim 1, further comprising receiving, from the user interface of the user device, an approval of the second meal plan option; and
adding the second meal plan option to a meal plan for a given period of time.

10. A method comprising:
determining a period of time;
determining, based at least in part on participant data associated with a participant and a health goal associated with the participant, one or more targets for the period of time;
presenting the one or more targets for the period of time on a display of a user device associated with the participant;
receiving consumption data associated with food items consumed during the period of time;
generating, based at least in part on the consumption data, progress data associated with the one or more targets; and
sending, via the user device, a notification associated with the progress data to the participant.

11. The method of claim 10, further comprising:
receiving a user selection of one or more selected targets from a presented list of possible targets for the period of time.

12. The method of claim 10, further comprising presenting on the user device one or more of:
relevant educational material associated with the one or more targets; and
relevant recommended actions associated with the one or more targets.

13. The method of claim 10, wherein determining the one or more targets for the period of time is based at least in part on one or more of:
prior progress data associated with a prior target associated with a second period of time prior to the period of time;
a prior health food score associated with a second period of time prior to the period of time;
a biology of the participant determined from sensor data associated with the participant or from one or more tests performed on the participant;
user inputs responsive to one or more questionnaires presented to the participant via the user device; or
a current diet of the participant determined based at least part on consumption data associated with the participant over prior period of time.

14. The method of claim 10, further comprising receiving the health goal as a user input from a user interface of a user device associated with the participant.

15. The method of claim 10, wherein sending the notification associated with the progress data to the participant is responsive to determining that the progress data meets or exceeds one or more thresholds.

16. The method of claim 10, wherein the target is a primary target and the method further comprises:
determining, based at least in part on the period of time, participant data associated with a participant, a health goal associated with the participant, and the primary target, a first intermediate target for a first subset of the period of time and a second intermediate target for a second subset of the period of time, the second subset different than the first subset;
receiving consumption data associated with food items consumed during the period of time;
determining, based at least in part on the consumption data, progress data associated with the first intermediate target or the second intermediate target; and
sending, via the user device, a notification to the participant in response to the progress data indicating a completion of the first intermediate target or the second intermediate target.

17. The method of claim 10, further comprising:
responsive to determining that the progress data meets or exceeds one or more progress thresholds and the period of time is less than or equal to a time threshold, determining an update to the target, and
sending, via the user device, a second notification associated with the update to the target.

18. The method of claim 10, wherein the participant data includes one or more of microbiome data associated with a gut of the participant, allergy data associated with the participant and health data associated with the participant.

19. A system comprising:
one or more processors; and
one or more non-transitory computer readable media storing instructions executable by the one or more processors, wherein the instruction, when executed, cause the one or more processors to perform operations comprising:
determining, based at least in part on participant data associated with a participant and a health goal associated with the participant, one or more targets for a period of time;
receiving consumption data associated with food items consumed during the period of time;
generating, based at least in part on the consumption data, progress data associated with the one or more targets; and
sending a notification associated with the progress data to the participant.

20. The system of claim 19, further comprising:
determining, based at least in part on the progress data, a completion metric associated with the one or more targets; and
wherein the notification includes the completion metric.
